SEQUENCE LISTING (2 sequences)

SEQ ID NO: 1 - protein, 11 aa, hemagglutinin antigen of Influenza strain
SFERFEIFPKE

SEQ ID NO: 2 - protein, 11 aa, hemagglutinin antigen of Influenza strain
SFEKFEIFPKT

(19) United States
(12) Patent Application Publication
(10) Pub. No.: US 2006/0156419 A1
(43) Pub. Date: Jul. 13, 2006

(54) Mouse model for autoimmune disorders

(75) Inventor: Andrew J Caton, Narbeth, PA (US)

Correspondence Address: HOWSON AND HOWSON, SUITE 210, 501 OFFICE CENTER DRIVE, FT WASHINGTON, PA, 19034, US

(73) Assignee: THE WISTAR INSTITUTE of ANATOMY and BIOLOGY, PHILADELPHIA, PA (US)

(21) Appl. No.: 10/532,971

(22) PCT Filed: Oct. 27, 2003
(86) PCT No.: PCT/US03/31519

§ 371 Date: Apr. 27, 2005

Related U.S. Application Data
(60) Provisional application No. 60/422,389, filed on Oct. 30, 2002.

(51) Int. Cl. A01K 67/027; C12N 5/06

(52) U.S. Cl. 800/3; 800/18; 435/354

(57) ABSTRACT

A non-human mammalian model of an autoimmune disorder co-expresses a major histocompatibility (MHC) class II-restricted T cell receptor (TCR) and a selected peptide that binds to the TCR. The selected peptide is selectively expressed by MHC class II positive antigen presenting cells (APC) of the mammal. Models with high penetrance of disease are those in which the selected peptide is a MHC class II-restricted T cell determinant that specifically binds with high affinity to the TCR. Models with low penetrance of disease are those in which the selected peptide binds with low affinity to the TCR. These models, which may be transgenic mammals, are used in method for identifying diagnostic and therapeutic markers and targets characteristic of an autoimmune disorder.

This invention was funded by grants AI24541, CA10185, 5T32CA09171, and 5T32EY07131 from the National Institutes of Health. The United States government has certain rights in this invention.

### BACKGROUND OF THE INVENTION

CD4+ T cells play a crucial role in the elaboration of many aspects of immune effector function. Some failure to establish CD4+ T cell tolerance to self-peptides likely underlies most autoimmune disease. For example, the identification of major histocompatibility complex (MHC) susceptibility alleles supports a role for CD4+ T cell recognition of self-peptides in a variety of autoimmune diseases, such as rheumatoid arthritis. Since the major function of MHC alleles is to present peptides derived from foreign and self antigens, susceptibility alleles potentially exert their effects by shaping the available T cell repertoire through presentation of target peptides for recognition by autoreactive CD4+ T cells, or both (Marrack et al. 2001 Nat. Med. 7: 899-905; and McDevitt 1998 Curr. Opin. Immunol. 10:677-81). How variations in the expression of peptides by MHC molecules can contribute to the systemic or organ-specific manifestations that present in different autoimmune diseases is at present not understood. Moreover, processes by which autoreactive CD4+ T cells become activated and promote the elaboration of pathologic effector functions remain poorly defined.

Rheumatoid arthritis (RA) is an example of an autoimmune disease that can exhibit both organ-specific and systemic manifestations (Lipsky, “Rheumatoid Arthritis” in Harrison's Principles of Internal Medicine, 1998 A. S. Fauci, et al., eds. New York, McGraw-Hill: pp. 1880-1888). The hallmark of RA is chronic inflammatory arthritis with progressive damage to articular structures including cartilage, bone, tendons and ligaments. RA patients can also develop extraarticular pathologies affecting vasculature and organs such as the lung. In addition, RA patients can produce antibodies to a variety of self-antigens, including immunoglobulin, filaggrin and the glycolytic enzyme glucose-6-phosphate isomerase (GPI) (Lipsky, 1998 cited above; Nogueira et al. 2001 Ann. Rheum. Dis. 60:882-7; Schaller et al. 2001 Nat. Immunol. 2:746-53).

Susceptibility to RA is substantially increased among individuals carrying specific MHC class II alleles (McCusker et al. 1991 Arthritis Rheum. 34:192-7 and Nepom 1998 Adv. Immunol. 68:315-32). Studies in rodent models suggest that CD4+ T cell responses to self-peptide(s) can be critical in disease pathogenesis. Immunization of susceptible rodent strains with collagen II induces collagen II-specific CD4+ T cells and a joint-targeted inflammatory process with features resembling RA (Holmdahl et al. 1990 Immunol. Rev. 118:193-232). In addition, transgenic mice bearing the K/B×N TCR specific for a peptide derived from GPI presented by I-A^{g7} spontaneously develop a destructive arthritis (Matsumoto et al. 1999 Science 286:1732-5). In this model, cognate interactions between K/BxN CD4+ T cells and GPI-specific B cells lead to the elaboration of highly arthritogenic GPI-specific antibodies.

While a wide variety of transgenic and knock-out mice have been found to develop autoimmunity, the disease either appears to be systemic or associated with inflammatory responses in the gut. Notably, however, neither I-A^{g7} mice that do not have K/BxN CD4+ T cells nor mice transgenic for human RA-susceptibility MHC alleles develop spontaneous inflammatory arthritis (Kouskoffet al. 1996 Cell 87:811-22 and Taneja et al. 1999 Immunol. Rev. 169:67-79).

There exists a continued need in the art for additional animal models, compositions and methods to enable the study of autoimmune disorders and the identification of biological targets for diagnosis, therapy and prophylaxis of autoimmune disorders, such as inflammatory arthritis.

### SUMMARY OF THE INVENTION

In one aspect, the invention provides a method for generating a non-human mammalian model of an autoimmune disorder. The method involves producing intermated progeny of a first and a second transgenic non-human mammal of the same species. The first mammal expresses a major histocompatibility (MHC) class II-restricted T cell receptor (TCR). The second mammal expresses a protein or peptide fragment thereof that binds to the TCR, which protein or peptide fragment thereof is selectively expressed by MHC class II positive antigen presenting cells (APC) of the mammal. Progeny that co-express the TCR and the peptide are selected by conventional means, such as PCR to detect the nucleotide sequences encoding the TCR and peptide. Progeny so selected develop an autoimmune disorder. In one embodiment, this method produces a high penetrance model of the autoimmune disorder. In another embodiment, the method produces a low penetrance model of the autoimmune disorder.

Another aspect of the present invention is a non-human mammalian model of an autoimmune disorder, produced by the method described above, including a non-human mammalian model of an autoimmune disorder having a high penetrance genotype, and a non-human mammalian model of an autoimmune disorder having a low penetrance genotype.

In a further aspect, the present invention provides a transgenic non-human mammal that expresses an MHC class II-restricted TCR and expresses a protein or peptide that binds to the TCR. The selected protein, peptide or a fragment thereof is selectively expressed by MHC class II positive APC of the mammal. This transgenic mammal develops the phenotypic symptoms of an autoimmune disorder. In one embodiment, this transgenic mammal exhibits high penetrance of the disorder. In another embodiment, the transgenic mammal exhibits low penetrance of the disorder.

In still a further aspect of the invention, a recombinant mammalian cell is provided that contains at least one transgene comprising a first nucleic acid sequence encoding and expressing an MHC class II-restricted TCR and a second nucleic acid sequence coding for a protein, peptide or fragment thereof that binds to the TCR. The second sequence is operably linked to a regulatory sequence that directs expression of the protein, peptide or fragment thereof by MHC class II positive APC.

In another aspect, the invention provides a method for producing a transgenic non-human mammalian model of an autoimmune disorder. In one embodiment, the method involves introducing at least one transgene comprising a first nucleic acid sequence that encodes an MHC class II-restricted TCR operably linked to regulatory sequences directing its expression; and a second nucleic acid sequence that encodes a protein, peptide or fragment thereof that binds to the TCR, operably linked to a sequence that directs expression of the protein, peptide or fragment thereof selectively to MHC class II positive APC into a fertilized egg of a mammal. Alternatively the transgene may be introduced into an embryonic stem cell of the mammal. Using the fertilized egg or embryonic cell to produce offspring in a pseudopregnant mammal results in transgenic offspring containing the transgene and having an autoimmune disorder.

In still another aspect, the invention provides a cell culture comprising cells derived from tissues of a transgenic non-human mammal described above.

In a further aspect, the invention provides a method of screening a compound for the ability to affect symptoms and/or reduce progression of an autoimmune disorder, e.g., inflammatory arthritis. A test compound is administered to a mammalian model of autoimmune disorder as described above and the severity of the symptom in the mammalian model is compared to that of a control mammal to which the test compound was administered or a control mammal to which no compound was administered. A compound that affects the symptom or reduces progression of the disorder is thereby identified. The model used in this assay may be one or more of the mammalian models or transgenic models described herein.

In another aspect, the invention provides a method of identifying a gene product responsible for the development of autoimmune disorders. In this method, expression levels of a selected gene product of a mammalian model of autoimmune disorder selected from those described herein is measured and compared with the expression of the same or analogous gene product in a control mammal. Any difference in the expression of the gene product is determined between the two mammals. The absence, upregulation or downregulation of the gene product between the model and the control is correlated with disease and identified.

In yet another aspect, the invention provides a method for identifying a biochemical marker of an autoimmune disorder by comparing the T cells or APC of a mammalian model with a high genotypic penetrance of the disorder with the T cells or APC of a mammalian model with a low genotypic penetrance of the disorder. A biochemical marker present on T cells or APC of one model is identified that is not present on the T cells or APC of the other model. The presence of a marker on the high penetrance model and its absence on the low penetrance model or the absence of the marker on the high penetrance model and its presence on the low penetrance model is an indicator of a high likelihood of the development of the autoimmune disorder.

Other aspects and advantages of the present invention are described further in the following detailed description of the preferred embodiments thereof.

### BRIEF DESCRIPTION OF THE FIGURES

FIG. 1 is a graph that illustrates the time course of joint inflammation, as measured in ankle width (mm) in TS1xHACII mice (gray circles), in individual TS1 and HACII mice (●); and in two sets of six 10 and 15 week-old TS1xHA104 mice (shaded circles). Solid lines connect the mean for each time-point. Statistical significance of differences between TS1xHACII mice and TS1, and HACII mice were determined using Mann-Whitney test, *P≦0.05, **P≦0.01.

FIG. 2A is a graph showing HA expression on unstimulated B cells (B220+) isolated directly ex vivo. Solid lines represent non-transgenic mice; bolded lines represent HACII mice; and dashed lines represent HA104 mice.

FIG. 2B is a graph showing HA expression on unstimulated dendritic cells (CD11c+) isolated directly ex vivo. The lines are the same as in FIG. 2A.

FIG. 2C is a graph showing HA expression on B-cells stimulated in vitro. The lines are the same as in FIG. 2A. The inset shows levels of MHC class II before (solid line) and following (bold line) in vitro stimulation.

FIG. 2D is a graph showing HA expression on dendritic cells (CD11c+) stimulated in vitro. The lines and insets are the same as in FIG. 2C.

FIG. 2E is a bar graph illustrating the results of ³H-thymidine incorporation measuring proliferation of LN cells from TS1 mice co-cultured with splenocytes from HA104, HACII or BALB/c mice with or without 1 μM S1 peptide. The inset of FIG. 2E is a graph which illustrates the proliferative response of TS1 LN cells to BALB/c splenocytes incubated with graded doses of S1 peptide.

FIGS. 3A and 3B are parallel graphs that illustrate the enhanced deletion and peripheral expansion of S1-specific CD4+ T cells in TS1xHACII mice. Thymi from TS1 mice (clear bar), TS1xHACII mice (shaded bar) and TS1xHA104 mice (dotted bar) were stained with antibodies to cell surface antigens, e.g., anti-CD4, anti-CD8 and 6.5. Thymic 6.5 levels were measured on CD4+ T cells (not shown) and numbers of CD4 positive (single positive, SP) T cells (CS4+SP) and thymic 6.5+ and CD4+ SP T cells (6.5+CD4+ SP) were measured. Symbols (●) represent individual mice and bars indicate mean frequencies (±s.d.). The frequency of 6.5+ CD4 SP thymocytes is significantly lower in TS1xHACII than TS1xHA104 mice (P=0.02, Mann Whitney test, indicated by asterisk).

FIGS. 3C and 3D are parallel graphs that illustrate the enhanced deletion and peripheral expansion of S1-specific CD4+ T cells in TS1xHACII mice. Spleens from TS1 mice (clear bar), TS1xHACII mice (shaded bar) and TS1xHA104 mice (dotted bar) mice were stained with anti-CD4, anti-CD8 and 6.5. Splenic 6.5 levels on CD4+ T cells were measured (not shown) and numbers of CD4 positive splenic T cells and 6.5+CD4+ thymic T cells were measured. Symbols (●) represent individual mice and bars indicate mean frequencies (±s.d.). The frequency of 6.5+CD4+ splenocytes is significantly higher in TS1xHACII than TS1xHA104 mice (P=0.01, Mann Whitney test, indicated by asterisk).

FIG. 4A is a graph which illustrates ³H-thymidine incorporation by measuring the proliferation of splenocytes from TS1 (●), TS1xHACII (∘) and TS1xHA104 (♦) mice in response to S1 peptide. Values shown are means of triplicate wells with standard deviation indicated.

FIG. 4B is a bar graph which illustrates proliferation of splenocytes from TS1, TS1xHACII, and TS1xHA104 mice in response to 1 μM S1 peptide, corrected for the number of 6.5+ CD4+ T cells in the initial culture as determined by flow cytometry. Data are representative of at least 3 experiments.

FIG. 4C is a graph that illustrates ³H-thymidine incorporation by measuring the proliferation of splenocytes from TS1 (●), TS1xHACII (∘) and TS1xHA104 (♦) mice in response to anti-CD3 antibody. Values shown are means of triplicate wells with standard deviation indicated.

FIG. 5A is a bar graph that illustrates enhanced antigen presenting cell activation in TS1xHACII mice by showing the numbers of splenic B cells (B220+) (shaded bars) and dendritic cells (CD11c+) (clear bars) from TS1, TS1xHACII and TS1×HA104 mice. Symbols (●) represent individual mice and bars represent the mean frequencies for each set of mice. The number of B cells and dendritic cells is significantly higher in TS1xHACII than TS1 mice (P=0.047 and 0.036, respectively, Mann Whitney test, indicated by asterisks).

FIG. 5B is a graph that illustrates MHC class II expression on splenic B220+cells from mice. Solids lines represent TS1 mice; bolded lines represent TS1xHACII mice; and dashed lines represent TS1xHA104 mice. The rightmost line represents HACII mice 4 days after adoptive transfer of lymph node (LN) cells from a TS1 mouse. Data shown are representative of at least six individual mice from three independent experiments.

FIG. 5C is a graph that illustrates CD86 (B7.2) expression on splenic B220+ cells from mice. Solids lines represent TS1 mice; bolded lines represent TS1xHACII mice; and dashed lines represent TS1xHA104 mice. The rightmost line represents HACII mice 4 days after adoptive transfer of LN cells from a TS1 mouse. Data shown are representative of at least six individual mice from three independent experiments.

FIG. 6A is a bar graph that provides ELISA analysis of sera from TS1, TS1xHACII and TS1xHA104 mice. Bars represent sera from individual TS1, HACII or HA104 mice (white bars), TS1xHA104 mice (grey bars), TS1xHACII mice (dotted bars), and positive control mice (black bars). Total serum IgG titers in TS1 (n=5), TS1xHACII (n=10) and TS1xHA104 (n=4) mice are expressed as the reciprocal of the dilution needed to give an absorbance at 405 nm of 0.35.

FIG. 6B is similar to FIG. 6A, but illustrates the levels of anti-HA, expressed as O.D. of a 1:100 dilution of sera.

FIG. 6C is similar to FIG. 6A, but illustrates the levels of IgM anti-IgG (rheumatoid factor), expressed as O.D. of a 1:100 dilution of sera.

FIG. 6D is similar to FIG. 6A, but illustrates the levels of anti-collagen IgG, expressed as O.D. of a 1:100 dilution of sera.

FIG. 6E is similar to FIG. 6A, but illustrates the levels of IgM anti-GPI, expressed as O.D. of a 1:100 dilution of sera.

FIG. 7A is a graph illustrating the binding affinity of CD4+ T cells of TCR transgenic mice, TS1, for the 11 amino acid S1 peptide derived from the hemagglutinin antigen of Influenza strain A/PR/8/34 (SFERFEIFPKE; SEQ ID NO: 1) and for the S1(SW) peptide that differs from the S1 peptide in two amino acids (SFEKFEIFPKT, SEQ ID NO: 2). The S1 peptide is the target peptide that is recognized by TS1 T cells in TS1xHACII mouse model of an autoimmune disorder resembling rheumatoid arthritis. The graph shows that the TS1 T cell receptor has a high affinity for the S1 peptide (▪) and low affinity for the S1(SW) peptide (∘).

FIG. 7B is a graph illustrating the binding affinity of CD4+ T cells of a different lineage of T cell receptor transgenic mice, TS 1(SW), for the S1 peptide (SEQ ID NO: 1) and the S1(SW) peptide (SEQ ID NO: 2). The graph shows that the TS1(SW) T cell receptor has a high affinity for the S1(SW) peptide (∘) and a low affinity for the S1 peptide (▪). The TS1(SW) T cell receptor has lower affinity for S1 shown by its needing around 30-fold higher concentrations of S1 peptide in order to reach half-maximal stimulation (measured here as proliferation in response to graded doses of the S1 peptide).

FIG. 8 is a graph showing ankle thickness versus weight in more than 8 week-old mouse models, in which ankle thickness results from the development of inflammatory arthritis. TS1xHA104 mice (□) are control mice that do not develop arthritis. The dashed lines show the interval in which 95% of the TS1xHA104 mice are predicted to lie based on statistical analysis of these data. The majority of the TS1xHACII mice (∘) exhibit ankle widths greater than the 95% prediction interval for TS1xHA104 mice. The age-matched cohort of TS1(SW)xHACII mice (open triangle) show only 4 mice exhibiting significant ankle swelling. The mice with ankle thicknesses above 3.4 mm have developed inflammatory arthritis as evidenced by immunohistochemical analyses of the joints. These data show that the overall avidity of the CD4+ T cell response to the S1 peptide can determine the degree of penetrance for inflammatory arthritis in these mouse models.

### DETAILED DESCRIPTION OF THE INVENTION

This invention provides non-human mammalian models of spontaneously developing autoimmune disorders, such as organ-specific or tissue-targeted autoimmune disorders, e.g., inflammatory arthritis. These models may be generated as described herein. These mammalian models of autoimmune disorder find use in research, and for diagnostic, therapeutic and prophylactic purposes, such as the identification of crucial biomarkers that participate/predict disease progression.

The present methods and compositions are based upon the discovery that autoreactive MHC class II-restricted T cells can precipitate a spontaneous, organ-specific, autoimmune disease in mammalian models when the model expresses an MHC class II-restricted T cell receptor and also expresses selectively by antigen presenting cells (APCs) an antigen that binds to the TCR. The development of the disorder is intimately linked to the cell types in which the antigen is expressed. Preferably, the antigen is a protein, peptide or fragment thereof that is a full or partial cognate of the TCR and is expressed selectively and endogenously by the APC of the model. High-level expression of the antigen by APCs in conjunction with a biasing of the CD4+ T cell repertoire toward specificity for that antigen, leads to the development of organ-specific autoimmunity. Surprisingly, organ-specific autoimmunity can be induced by a CD4+ T cell response to an antigen that is not directly related to the target organ. The development of autoimmunity in this model is not due to some unknown specificity of the TCR for a self-antigen. As illustrated by the Examples below, a similar animal model with the same antigen expressed systemically, does not develop the symptoms of the autoimmune disorder, e.g., inflammatory arthritis. An important parameter that dictates the penetrance of the disease is the avidity of the T cell response to the antigen.

I. Definitions

The term “non-human mammalian model” as used herein means all mammals, except humans, bearing genetic information, i.e., a “transgene”, received, directly or indirectly, by deliberate genetic manipulation at a subcellular level. Transgenic animals include animals in which the genetic information was introduced into a germ line cell, thereby conferring the ability to transfer the transgene to offspring. Also encompassed by this term are offspring of the transgenic animals described herein provided by using classical breeding techniques or in vitro fertilization. These models are characterized by co-expression of a TCRs and an antigen that binds the TCR, which latter antigen is expressed by the animal's APCs. For convenience, the following discussion will refer to transgenic mice or intermated progeny of transgenic mice. However, it should be understood that other non-human mammals are similarly useful.

As used herein, a “transgene” is a DNA sequence introduced into the germ line of a non-human animal by way of human intervention, such as by the methods described herein. Transgenes of the present invention encode a MHC class II-restricted TCR under the control of suitable regulatory sequences with direct high level expression of the TCR and/or encode an antigen that binds thereto under the control of regulatory sequences that direct high level expression of the antigen by APCs.

The term “cognate” or “cognate antigen” as used herein means a naturally-occurring, recombinant or synthetic major histocompatibility (MHC) class II complex-restricted T cell determinant (i.e., protein or peptide) to which an MHC class II-restricted TCR binds preferentially at high avidity, or alternatively, the peptide or antigen which the TCR recognizes as an agonist. Generally, the cognate protein comprises a peptide fragment that binds to the TCR. Such peptide fragments or determinants recognized by the TCR are generally about 7 to about 23 amino acids in length. In some embodiments, the T cell determinant is from about 12 to about 16 amino acids in length. According to this disclosure, the cognate can be a protein or peptide that is not associated with any autoimmune disorder or not related to the target organ for organ-specific autoimmune disorders.

The term “binding peptide” as used herein refers to a naturally-occurring, recombinant or synthetic major histocompatibility (MHC) class II complex-restricted T cell determinant (i.e., antigen or peptide) to which an MHC class II-restricted TCR binds at low avidity. Low avidity binding permits the use of the peptide to develop low penetrance mammalian models. High affinity binding between the TCR and the peptide permits the development of high penetrance mammalian models according to this invention.

The phrase “selectively or endogenously expressed by MHC class II positive APCs” as used herein means that the referenced peptide is expressed solely by the APCs and by no other cells in the mammal. This manner of expression is in contrast to systemic expression, in which the reference peptide is expressed by numerous cells of the mammal, which may include APCs.

The term “antigen presenting cells” or “APC” refers to B cells, macrophages, dendritic cells, or any cell that can be induced to express MHC class II antigen in response to stimuli, and combinations thereof. Examples of such stimuli include cytokines, particularly pro-inflammatory cytokines, such as gamma interferon.

The term “high affinity” or “high avidity” as used herein is meant to refer to the level of binding of a TCR to its specific or preferred cognate peptide or agonist. When that cognate peptide is altered, the binding of the TCR is reduced. High affinity or high avidity between two ligands may generally be described as half maximal stimulation at between 0.01 to 1 micromolar peptide. Such value may be determined in conventional assays, such as proliferation assays. See Example 1 below.

The term “low affinity” or “low avidity” as used herein is meant to refer to the level of binding of a TCR to a peptide that is reduced relative to its binding affinity or avidity to its specific cognate peptide. Low affinity or low avidity between two ligands may generally be described as half maximal stimulation of greater than 1 micromolar peptide. Such value may be determined in conventional assays, such as proliferation assays. See Example 1 below.

The term “high penetrance” as used herein is meant to refer to the degree to which a genotype creates a predictable phenotype, i.e., in this case, the spontaneous development of an autoimmune disorder. This term as used herein relates to an inheritance of the genotype comprising an MHC class II restricted TCR, and its cognate peptide that binds with high affinity to the TCR and is expressed by APC. This genotype results in at least 50% of progeny having the phenotype comprising the spontaneous development of autoimmune disorder symptoms, such as symptoms resembling inflammatory arthritis or rheumatoid arthritis. In some models this genotype results in at least 70% or more of progeny having the phenotype.

The term “low penetrance” as used herein is meant to refer to a reduced degree to which a genotype creates a predictable phenotype, e.g., the spontaneous development of an autoimmune disorder. This term as used herein relates to an inheritance of the genotype comprising an MHC class II restricted TCR, and a peptide having a low binding affinity or avidity to the TCR that is expressed by APC. Inheritance of this genotype can result in less than 20% of progeny having the phenotype comprising symptoms resembling inflammatory arthritis or rheumatoid arthritis. In some models this genotype results in less than 10% or less of progeny having the indicated phenotype.

The term “autoimmune disorder or disease” as used herein refers in one embodiment to inflammatory arthritis. The Examples below describe models of an inflammatory arthritis similar to human rheumatoid arthritis that consists of one or more of the following symptoms, without limitation, inflamed joints with bone resorption, mononuclear cell infiltrates and pannus formation, bone erosion, bone remodeling, vasculitis, interstitial pneumonitis in the lung, anti-nuclear antibodies in serum, and weight loss, and further includes increased severity in females. Still other autoimmune disorders that may develop in models of this invention include other symptoms that are listed in the clinical diagnosis of autoimmune disorders such as Crohn's disease, multiple sclerosis, rheumatoid arthritis, systemic lupus erythematosis, inflammatory bowel disease, nephritis, autoimmune thyroiditis, reactive arthritis, ankylosing spondylitis, scleroderma, polymyositis, dermatomyositis, psoriasis, Wegener's granulomatosis, ulcerative colitis, Sjogren's syndrome, sarcoidosis, insulin dependent diabetes mellitus (IDDM), as well as collections of symptoms that resemble any of the above-listed disorders. Additionally, tissue-targeted automimmune diseases include autoimmune diseases in which disease develops selectively in a single tissue, e.g., joints or lungs, among other disorders.

For the common definitions of other terms of art, see, e.g., texts such as C. A. Janeway et al (eds), Immunobiology, Garland Publishing, 5^{th} edition, 2001, ISBN 081533642X.

II. Mammalian Models of Spontaneous AutoImmune Disorder

A. Progeny of Intermating Two Transgenic Mammals

The invention provides a method for producing a non-human mammalian model of an autoimmune disorder by providing in a selected mammal the nucleic acid sequences necessary to express a major histocompatibility (MHC) class II-restricted T cell receptor (TCR) and the nucleic acid sequences necessary to express a selected peptide that binds to the TCR. The latter nucleic acid sequence also contains the necessary sequences for directing expression of the selected peptide selectively by MHC class II positive antigen presenting cells (APC) of the mammal.

Thus, in one aspect of the invention, a method for generating suitable animal models involves producing intermated progeny of two transgenic non-human mammals of the same species. The first mammal expresses a major histocompatibility (MHC) class II-restricted TCR. A number of such mammals are known, including the TS1 mouse that expresses a transgene-encoded MHC class II restricted TCR specific for the major I-Ed-restricted determinant S1 (SEQ ID NO: 1) of influenza strain A/PR/8 HA, described in J. Kirberg et al, 1994 J. Exp. Med., 180:25-34, and other references. See also the transgenic TCR(SW) mouse described in Jordan et al., 2001 Nat. Immunol. 2:301-6. Still other exemplary mouse models expressing MHC class II-restricted TCRs include, inter alia, the mouse expressing the TCR for cytochrome C (L. J. Barg et al, 1988 Mol. Cell. Biol, 8(12):5459-5469); a mouse model expressing the TCR for hemoglobin (C. B. Williams et al, 1999 J. Exp. Med., 179(5):1531-44), a mouse model expressing the TCR for hen egg lysozyme (W. Y. Ho et al, 1994 J. Exp. Med., 179(5):1539-49). It is anticipated that one of skill in the art may generate other suitable mammalian models expressing mammalian MHC class II-restricted TCR by access to knowledge available in the art.

The second mammal expresses a selected peptide or agonist that binds to the TCR. This peptide is selectively expressed by MHC class II positive antigen presenting cells (APC) of the mammal. The second mammal contains a nucleic acid sequence encoding the peptide that is operably linked to regulatory sequences that enable expression of the peptide by the MHC class II positive APC. These regulatory sequences may include the sequences that regulate expression of certain genes that are selectively presented by APC, sequences that direct the expression of different genes involved in antigen presentation, MHC class II promoters or analogous sequences involved in the expression of antigens on APCs in a variety of mammals.

For example, an MHC class II promoter used in the models exemplified herein is the MHC class II I-Eα gene promoter. Similar mouse promoter sequences are those non-MHC class II sequences involved in expression of the invariant chain or the H2-M promoter. Still other promoters that drive expression of antigens by the APCs include the Dec205 promoter and the Cd11c promoter. Other sequences analogous to these or known for expression of antigens on APCs in other mammals, e.g., transferrin, may be similarly employed, provided that they have the ability to target expression of the peptide to the APC.

Similarly fragments of the above regulatory sequences that direct expression of an antigen by the APCs may be employed. Such functional fragments of the MHC class II antigens are peptides of at least 7 consecutive amino acids in length from the MHC class II antigen that retain the targeting ability. In one embodiment such fragments are at least 10 amino acids in length. In other embodiments, such fragments are about 20 to 30 amino acids in length. Such regulatory sequences and functional fragments may also be synthetic, recombinant or modified sequences that share the function of directing the peptide for expression by MHC class II positive APC.

The selected peptide itself may be any peptide amino acids in length that binds with high or low affinity to the TCR, when the peptide is expressed by APCs. In some embodiments, the peptide is embedded in a protein, e.g., HA. In other embodiments, the peptide is between about 7 to about 23 amino acids in length. Proteins that contain such suitable peptides may be selected from within any number of antigenic sequences, including, without limitation, the proteins that are recognized by the TCRs listed above, e.g., hemagglutinin, hemoglobin, cytochrome C, and lysozyme. Still other peptides that bind to TCRs may be selected from among many known and available sequences and such selection is within the ability of the person of skill in the art given the teachings of this disclosure. See, A. Rudensky and C. Janeway, 1993 Chem. Immunol., 57:134-15; A. Rudensky et al, 1991 Nature, 353:662-6271 also, Janeway et al (eds), Immunobiology, cited above, all incorporated by reference herein.

By “operably linked” for purposes of the nucleic acid sequence encoding the TCR binding peptide is meant that the peptide-encoding sequence is associated with the regulatory nucleic acid sequence in a conventional manner suitable for the regulatory sequence to direct expression of the peptide by the APC. The assembly of the peptide encoding sequence in the appropriate position vis-à-vis the regulatory sequence or promoter involves knowledge available to one skilled in the art.

An exemplary “second mammal” for intermating with the TS1 transgenic mouse is the novel HACII transgenic mouse. As described in more detail in Example 1, the HACII mouse was generated by introducing into germ cells of Balb/c x C57/B16 parental strain, a modified pDOI-5 plasmid. The pDOI-5 plasmid (Kouskoff et al. 1993 J. Immunol. Methods 166:287-91) contains a portion of the MHC class II I-Eα gene promoter and splicing/polyadenylation signals. To generate HACII transgenic mice, a cDNA copy of the HA gene of influenza virus A/PR/8134 (PR8) was inserted into the unique EcoRI site of the plasmid pDOI-5. The HA gene is thereby linked to and its expression directed by the portion of the MHC class II I-Eα gene promoter and splicing/polyadenylation signals provided by the plasmid. This linkage of the HA gene to the MHC class II promoter directs expression of the HA to B cells and CD11c+ dendritic cells.

The generation of similar transgenic mammals that express a peptide by MHC class II positive APCs including suitable regulatory sequences other than the MHC class II I-Eα gene is within the skill of the art. See, for example, the descriptions below on the construction of transgenic animals and the other references cited herein.

The mating of two such transgenic animals produces progeny tha2t inherit the above-described transgenes by Mendelian inheritance. Progeny that co-express the TCR and the selected peptide are selected and identified by conventional means. As one example, the polymerase chain reaction technique can be employed with primer sequences that can hybridize to portions of the HA gene and MHC promoter and to the TCR sequence to detect the presence of these essential nucleotide sequences in progeny. Progeny so selected develop an autoimmune disorder, e.g., inflammatory arthritis and symptoms similar to human rheumatoid arthitis.

In one embodiment of this method, a high penetrance model of the autoimmune disorder is generated. To produce the high penetrance model of disease, the selected peptide expressed by the second mammal selectively by APCs is a cognate of the TCR, that is, a naturally-occurring, recombinant or synthetic MHC class II-restricted T cell determinant that specifically binds with high affinity to the TCR. For example, the intermating of TS1xHACII mice produces a high penetrance model, because progeny of this intermating of transgenic mice that express the TS1 TCR and its cognate peptide S1, presented by APC of the mouse.

In another embodiment, the method produces a low penetrance model of the autoimmune disorder by using as the selected peptide expressed by the second mammal selectively by APCs, a naturally-occurring, recombinant or synthetic protein or peptide fragment that binds with low affinity to the TCR. For example, a low penetrance model could be a cross between TS1 and a mouse expressing a mutant S1 peptide on APCs. Alternatively, a low penetrance model is described in Example 8, by intermating a TCR model expressing an MHC class II-restricted TCR with high affinity for a synthetic mutant S1 analog (TS1(SW)) with the HACII mouse, that expresses native S1. The low affinity binding between the TCR and peptide produces a low penetrance model of an autoimmune inflammatory arthritis.

Following this method, intermated progeny of other pairs of TCR expressing animals with other animals expressing a low or high-affinity agonist of the TCR produce resulting progeny that develop an autoimmune disorder. In another embodiment, this method may also be employed to produce systemic inflammatory responses affecting multiple tissues and organ systems in the animals. Such system responses may be seen in up to 15% of the progeny. In still another embodiment, tissue-targeted or tissue-specific autoimmune disorders are produced at about 85% of the progeny, e.g., joints, lungs and other tissues, depending upon the TCR and peptides employed in the method. In still another embodiment the method produces tissue-targeted automimmune disease resembling rheumatoid arthritis as a manifestation of a systemic anti-self immune response.

B. Novel Transgenic Mammalian Models of the Invention

Still a further aspect of this invention are novel transgenic non-human mammalian models that express a major histocompatibility (MHC) class II-restricted T cell receptor (TCR) and also express a peptide that binds to the TCR. This peptide is selectively expressed by MHC class II positive antigen presenting cells (APC) of the mammal. This transgenic mammal develops the phenotypic symptoms of an autoimmune disorder.

Methods for producing transgenic animals are known to those of skill in the art. In one embodiment a method for producing a transgenic animal of the invention involves directly injecting the transgene DNA into nuclei of fertilized eggs. See, e.g., Brinster et al, 1986 J. Anim. Sci., 63(1):269-78. In another method, i.e., the embryonal stem cell (ES) method, ES cells are obtained from pre-implantation embryos and cultured in vitro Evans et al, 1981 Nature, 292:154-156). Transgenes are introduced into the ES. Still other known methods of generating transgenic animals may be employed in this invention.

Because the transgene must be delivered or introduced to a fertilized egg or an embryonic cell in the methods discussed herein, a nucleic molecule encoding the TCR is employed in the methods of this invention. One or more transgenes are constructed which contain a nucleic acid sequence that expresses a major histocompatibility (MHC) class II-restricted TCR (i.e., a transgene). In addition to DNA, and occasionally RNA sequences encoding the components of the transgene described above, the nucleic acid molecule also contains suitable regulatory sequences directing expression of the TCR in a parent cell. For example, suitable transgenes were utilized in the development of the TS1 and TS1(SW) transgenic mice models. The nucleic acid molecule which encodes the transgene and introduces it into the fertilized egg or ES cell can be a plasmid or a viral or non-viral recombinant vector. Both viral vectors and non-viral vectors may be used (including non-viral methods of delivery of a nucleic acid molecule into a cell).

One type of recombinant vector is a recombinant viral vector. A variety of viral vector systems are known in the art. Examples of such vectors include recombinant adenoviral vectors, herpes simplex virus (HSV)-based vectors, adeno-associated viral (AAV) vectors, hybrid adenoviral/AAV vectors, recombinant retroviruses or lentiviruses which are constructed to carry or express a selected nucleic acid composition of interest.

Retrovirus vectors that can be employed include those described in EP 0 415 731; International Patent Publication Nos. WO 90/07936; WO 94/03622; WO 93/25698; and WO 93/25234; U.S. Pat. No. 5,219,740; International Patent Publication Nos. WO 93/11230 and WO 93/10218; Vile and Hart, 1993 Cancer Res. 53:3860-3864; Vile and Hart, 1993 Cancer Res. 53:962-967; Ram et al., 1993 Cancer Res. 53:83-88; Takamiya et al., 1992 J. Neurosci. Res. 33:493-503; Baba et al., 1993 J. Neurosurg. 79:729-735; U.S. Pat. No. 4,777,127; GB Patent No. 2,200,651; and EP 0 345 242. Examples of suitable recombinant retroviruses include those described in International Patent Publication No. WO 91/02805.

Alphavirus-based vectors may also be used as the nucleic acid molecule encoding the transgene. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532). Representative examples of such vector systems include those described in U.S. Pat. Nos. 5,091,309; 5,217,879; and 5,185,440; and International Patent Publication Nos. WO 92/10578; WO 94/21792; WO 95/27069; WO 95/27044; and WO 95/07994.

Examples of adenoviral vectors include those described by Berkner, 1988 Biotechniques 6:616-627; Rosenfeld et al., 1991 Science 252:431-434; International Patent Publication No. WO 93/19191; Kolls et al., 1994 PNAS 91:215-219; Kass-Eisler et al., 1993 PNAS 90:11498-11502; Guzman et al., 1993 Circulation 88:2838-2848; Guzman et al., 1993 Cir. Res. 73:1202-1207; Zabner et al., 1993 Cell 75:207-216; Li et al., 1993 Hum. Gene Ther. 4:403-409; Cailaud et al., 1993 Eur. J. Neurosci. 5:1287-1291; Vincent et al., 1993 Nat. Genet. 5:130-134; Jaffe et al., 1992 Nat. Genet. 1:372-378; and Levrero et al., 1991 Gene 101:195-202. Exemplary adenoviral gene therapy vectors include those described in International Patent Publication Nos. WO 94/12649; WO 93/03769; WO 93/19191; WO 94/28938; WO 95/11984 and WO 95/00655. Other particularly desirable adenoviral vectors include those derived from chimpanzee adenoviruses, such as those described in U.S. Pat. No. 6,083,716.

Another viral vector is based on a parvovirus such as an adeno-associated virus (AAV). Representative examples include the AAV vectors disclosed by Srivastava in International Patent Publication No. WO 93/09239, Samulski et al., 1989 J. Virol. 63:3822-3828; Mendelson et al., 1988 Virol. 166:154-165; and Flotte et al., 1993 PNAS 90:10613-10617. Other particularly desirable AAV vectors include those based upon AAV1; see, International Patent Publication No. WO 00/28061, published May 18, 2000. Other desirable AAV vectors include those which are pseudotyped, i.e., contain a minigene composed of AAV 5′ ITRS, a transgene, and AAV 3′ ITRs packaged in a capsid of an AAV serotype heterologous to the AAV ITRs. Methods of producing such pseudotyped AAV vectors are described in detail in International Patent Publication No. WO01/83692.

The nucleic acid molecule of the invention may also include non-viral vectors or methods for delivery of the sequence encoding the transgene to the parent cell according to this invention. A variety of non-viral vectors are known in the art and may include, without limitation, plasmids, “naked” DNA and DNA condensed with cationic lipids or polymers. In one embodiment, the polymers may include traditional polymers and non-traditional polymers such as cyclodextrin-containing polymers and protective, interactive noncondensing polymers, among others. The “naked” DNA and DNA condensed with cationic lipids or polymers are typically delivered to the cells using chemical methods. A number of chemical methods are known in the art for cell delivery and include using lipids, polymers, or proteins to complex with DNA, optionally condensing the same into particles, and delivering to the cells. Another non-viral chemical method includes using cations to condense DNA, which is then placed in a liposome and used according to the present invention. See, C. Henry, 2001 Chemical and Engineering News, 79(48):35-41.

Whether the nucleic acid molecule is a viral vector or non-viral vector, it may optionally contain regulatory sequences in addition to the sequences encoding the transgene. For example, such regulatory sequences comprise a promoter which drives expression of the transgene. For expression of the TCR, suitable promoters and regulatory elements are preferably obtained from the native T cell, i.e., the T cell specific regulatory sequences. For example, the regulatory sequences used to create the TS1 mouse model, the TS1(SW) mouse model and other TCR-expressing transgenic mice may be employed. These native sequences that drive expression of T cell antigens may be isolated from genomic clones of T cells. Generally, in isolating the sequences from genomic clones, one must isolate about 20 kb from the T cells, in which about 15 kb upstream of the about 5 kb exon coding for the TCR comprises the T cell regulatory sequences. Alternatively, such sequences are available in plasmids such as pTα and pTβ (V. Kouskoff et al, 1995 J. Immun. Meth., 180(2):273-80) or similar plasmids that contain all sequences that drive TCRs (see e.g., ATCC Accession No. 59584). It may also be possible to employ regulatory sequences selected from among known constitutive promoters, inducible promoters, tissue-specific promoters and others. See, e.g., the promoters listed in International Patent Publication No. WO01/83692 for additional lists of known promoters.

Other regulatory sequences that may be present in a nucleic acid molecule of this invention include, among others, epitope tags, nuclear localization sequences, IRES elements, TATA elements, polyadenylation sites, restriction enzyme cleavage sites, selectable markers and the like. Selection of promoters and other common vector elements are conventional and many such sequences are available (see, e.g., Sambrook et al, Molecular Cloning. A Laboratory Manual, Cold Spring Harbor Laboratory, New York, 1989 and references cited therein at, for example, pages 3.18-3.26 and 16.17-16.27 and Ausubel et al., Current Protocols in Molecular Biology, John Wiley & Sons, New York, 1989).

To generate the transgenic models of this invention a second nucleic acid molecule, which is can be on the same molecule as the TCR-expressing transgene, or on a separate nucleic acid molecule, contains a nucleic acid sequence that encodes a selected peptide that binds to the TCR. This peptide expressing transgene is operably linked to a sequence that directs expression of the peptide selectively to MHC class II positive APCs. Provided that this transgene is linked to a sequence directing its expression by the APCs (as described above for non-transgenic models), the other regulatory sequences discussed above for use in expressing the TCR transgene may be incorporated into the molecule.

According to several known methods for making transgenic animals, the transgene(s) is recombinantly introduced into a fertilized egg of the mammal, or an ancestor of the mammal, or into an ES cell. For example, the one or more nucleic acid molecules containing the transgenes for the TCR and the binding peptide are efficiently introduced into a fertilized egg or into the ES cells by DNA transfection. Such transformed ES cells can thereafter be combined with blastocysts from a non-human animal. The ES cells then colonize the embryo and contribute to the germ line of the resulting chimeric animal (see, e.g., R. Jaenisch, 1988 Science, 24:1468-1474; and Hogan et al., Manipulating the Mouse Embryo. A Laboratory Manual, Cold Spring Harbor, N.Y.: Cold Spring Harbor Press (1986)). Thereafter the ES cells are injected into an embryo of the mammal.

The fertilized egg which has been directly injected with the transgenes or the embryo from the ES method described above is transplanted into a pseudopregnant mammal. After allowing the fertilized egg or embryo to develop to term, at least one transgenic offspring containing the TCR and peptide/MHC class II promoter sequences is identified that develops spontaneous, organ-specific autoimmunity.

Thus, in one embodiment the spontaneous autoimmune disorder phenotype is conferred by at least one transgene contained in somatic and germ cells of the mammal which directs the co-expression of the selected peptide selectively by its APC and the TCR. As with the non-transgenic models described above, the transgenic mammal exhibits high penetrance of the disorder when the transgene encoding the selected peptide is a sequence encoding an MHC class II-restricted T cell determinant that specifically binds with high affinity to the TCR, and wherein the mammal. In another embodiment, the transgenic mammal exhibits low penetrance of the disorder when the transgene encoding the selected peptide is a protein or peptide fragment that binds with low affinity to the TCR.

The transgenic non-human mammal is fertile and can produce progeny with the same genotype, when bred with other mammals of the same species or with non-transgenic mice. Back-crossing may be employed to stabilize the genome. The transgenic mice that develop spontaneous, organ-specific autoimmunity can be identified by genotyping by conventional methods, such as PCR.

Clearly one of skill in the art may employ other methods for generating transgenic models, which may include the use of preferred promoters and regulatory sequences and any set of MHC class II-restricted TCR and cognate peptide or low affinity binding peptide, and any suitable regulatory sequences, including MHC class II promoters, fragments and antigens which direct expression of the binding or cognate peptide to the mammals' APCs. Selection of these elements is all within the art and given this disclosure, generation of a variety of different transgenic and intermated progeny exhibiting the phenotype of spontaneous development of autoimmune disorder may be produced.

III. Cells of the Invention

Recombinant mammalian cells containing one or more of the transgenes described above as useful for generating the transgenic mammals of this invention may be prepared by conventional methods. Particularly desirable cells are selected from among any mammalian species, such as human, monkey, mouse, rat, rabbit, and hamster, among others. Exemplary cells for use in this invention include specifically, without limitation, primary fibroblast, hepatocyte and myoblast cells derived from mammals cells; various murine cells, e.g., 10T1/2 and WEHI cells, African green monkey cells such as VERO, COS1, COS7, BSC1, BSC 40, and BMT 10, and human cells such as W138, MRC5, A549, human embryonic retinoblast (HER), human embryonic kidney (HEK), human embryonic lung (HEL), and TH1080 cells. Other appropriate cells include 293 cells (human embryonic kidney cells which express adenoviral E1a and E1b proteins), 911, PER.C6 cells (human embryonic retinoblast cells that express adenoviral E1; see WO 97/19463), GH329 cells (a cell line that expresses adenoviral E1); 27-18 cells, 84-31 cells (293-based cells that express adenovirus E1a, E1b and E4 (G. Gao, J. Virol., 70(12):8934-8943 (1996)), 10-3 cells (293-based cells that express adenovirus E1a, E1b and E4ORF6 (G. Gao, J. Virol, (1996)), 3T3 cells (mouse embryonic fibroblast cell line), NIH3T3 cells (subline of 3T3 cells), HepG2 cells (human liver carcinoma cell line), Saos-2 cells (human osteogenic sarcomas cell line), HuH7 cells or HeLa cells (human carcinoma cell line). Still other suitable mammalian cells include 10T1/2, BHK, MDCK, Saos, C2C12, L cells, HT1080, CHO, BKH, and MDCK A nucleic acid molecule expressing the above-defined transgenes is introduced into a parent cell. As described above, the elements of the nucleic acid molecule encoding the transgenes and the identity of the parent cell may all be selected by one of skill in the art. By “introducing” the nucleic acid into the cell is meant delivering the nucleic acid molecules to the cells in any manner known to one in the art, including, without limitation, transfection, infection, electroporation, sonopoation, liposome delivery, membrane fusion techniques, high velocity DNA-coated pellets, viral infection and protoplast fusion, or particle bombardment. However, other methods known by those skilled in the art may be utilized.

In one embodiment, the nucleic acid molecules may be transfected into the host cell and exist stably in the cell as an episome. In another embodiment, the sequences encoding the transgenes are stably integrated into the genome of the cell. Another embodiment has the sequences transiently expressed in the host cell.

The above-described transgenes and nucleic acid molecules, its various components parts and the recombinant cells described above may be constructed recombinantly using conventional molecular biology techniques, site-directed mutagenesis, genetic engineering or PCR, and the like by utilizing the information provided herein. For example, methods for producing the above-identified modifications of the sequences include mutagenesis of certain nucleotides and/or insertion or deletion of nucleotides, or codons, thereby effecting the polypeptide sequence by insertion or deletion of, e.g., non-natural amino acids. Such methods are known and may be selected by one of skill in the art. Similarly, methods for producing plasmid, other non-viral vector constructs or viral vector constructs encoding the TCRs, binding peptides, and/or any other molecules used herein are well-known in the art, as are methods for using expression systems to produce the proteins.

The transgenic non-human mammalian models or intermated non-human mammalian models described herein can also be used as a source of cells for cell culture. Cells of tissues carrying the above-identified transgenes can be cultured, by standard tissue culture techniques.

The recombinant cells and the cell cultures derived from the mammalian models can be used for a variety of research and/or diagnostic purposes that will be readily apparent to those of skill in the art. As one example, these cells and cell cultures may be useful for identifying biomarkers, and screening diagnostic or therapeutic compounds, etc., as described hereinbelow.

IV. Methods of Using the Mammalian Models and Cells

The transgenic and intermated mammalian models of the invention as well as the tissue cell cultures obtained therefrom have a variety of uses which include research uses for autoimmune disorders, screening for drugs useful in diagnosing and treating autoimmune disorders such as inflammatory arthritis, and associated conditions.

In one embodiment, these models may be employed in a method of screening a compound for the ability to effect or delay symptoms of an autoimmune disorder. Such a method would involve administering a test compound to a mammalian model of autoimmune disorder as described herein. Any effect of the compound upon the severity of the symptoms or the development of the disorder in the mammalian model would be compared to the results obtained when the same compound is administered to a control model without the autoimmune disorder or when no compounds is administered to the same model. For example, the impact on the autoimmune symptoms in the models of this invention may be impacted by administration of test compounds, which can include nucleic acid molecules (e.g., DNA vaccines, plasmids and the like), proteins or synthetic chemical pharmaceutical or diagnostic compounds. These compounds can inhibit inflammatory responses or otherwise interfere with the immune system. Exemplary protein test compounds include, without limitation an isolated antibody, a monoclonal antibody, a recombinant antibody, a chimeric antibody, a synthetic antibody, an antibody fragment, a cytokine, and a chemokine. The antibody may include without limitation any antibody that inhibits an inflammatory response or receptor, e.g., anti-TNF-γ, an anti-nuclear antibody, or anti-cytokine antibodies, such as anti-IL-12 or anti-IL2. Similarly, antibody to TCR may be used as a test compound on the models. Other possible test compounds include any protein involved in inflammation, such as C-reactive protein or anti-tumor necrosis factor gamma. Test compounds that are nucleic acid molecules may be viral vectors or plasmid vectors encoding any number of other substances.

In another embodiment, the invention provides a method of identifying a gene product responsible for the development of an autoimmune disorder. For example, expression levels of various gene products expressed in a mammalian model of autoimmune disorder may be identified. The level of expression of a gene product in the model may be compared with the expression of the same or analogous gene product in a control mammal. Any difference in the expression of the gene product, for example, the absence of the gene product, the upregulation of the gene product, or the downregulation of the gene product in the model as compared with a control would provide an indication that the gene product was a biomarker of the disorder.

In any assay method or screening method, a suitable control mammal may be a sibling of the model that does not demonstrate any or all of the symptoms of the autoimmune disorder, or a low penetrance model of the disorder, an unrelated mammal without disease, one of the parental mammals of an intermated model, or a model that expresses the same peptide as does the model of the invention, but expresses it systemically.

Still another method of use of the models of this invention includes a method for identifying a biochemical marker of an autoimmune disorder. The T cells, particularly the helper T cells or the APC of a mammalian model with a high genotypic penetrance of the disorder can be isolated and compared with the T cells or APC of a mammalian model with a low genotypic penetrance of the disorder. The methods of analysis of these cells can be accomplished by use of conventional techniques such as polymerase chain reaction, Southern hybridization, and the like. A biochemical marker present on T cells or APC of one model that is not present the T cells or APC of the other model is a candidate biochemical marker of that autoimmune disorder. The presence of the marker on the high penetrance model and its absence on the low penetrance model may indicate that the marker is related to the development of the disorder. Alternatively, the absence of the marker on the high penetrance model and its presence on the low penetrance model may indicate that the absence is an indicator of a high likelihood of the development of the autoimmune disorder.

Alternatively, such models of autoimmune disorder may be used to screen for novel anti-arthritis compounds, for example.

V. The Examples

The following examples are provided to illustrate the invention and do not limit the scope thereof. One skilled in the art will appreciate that although specific reagents and conditions are outlined in the following examples, modifications can be made which are meant to be encompassed by the spirit and scope of the invention.

The following examples demonstrate that when a nominal peptide, HA S1, is expressed selectively by antigen presenting cells (APC) in an intermated progeny of a model expressing a high affinity TCR for S1 (TS1xHACII mice), inflammatory arthritis develops despite profound deletion of HA-specific thymocytes in 20 week-old mice. Yet, HA-specific CD4+ T cells that escape central deletion become activated in the periphery. APCs also become activated, and the mice develop an inflammatory and destructive arthritis. Importantly, CD4+ T cell recognition of HA induces arthritis only when HA is synthesized endogenously by APCs. Mice in which HA is expressed diffusely or systemically (e.g., TS1xHA104 mice), but not synthesized directly by APCs themselves, exhibit other autoimmune manifestations without developing arthritis.

The findings of these examples demonstrate that inflammatory arthritis, such as human rheumatoid arthritis, can result from an autoreactive CD4+ T cell response to APC-derived self-peptide(s) in a process that has similarities to allorecognition. Thus protection against autoimmune disease need not be determined by the efficiency with which autoreactive thymocytes are deleted, because both the extent of thymic deletion and the severity of autoimmune disease were enhanced in TS1xHACII relative to TS1xHA104 mice.

That TS1xHACII mice develop severe autoimmune disease despite profound deletion of S1-specific thymocytes reflects processes acting in the periphery, during T cell repertoire formation. In the periphery, rare 6.5+CD4+ T cells that evade deletion in TS1xHACII mice can initiate the disease process by causing APCs to express elevated levels of the HA S1 peptide. APCs from HACII mice up-regulate cell surface expression of HA in response to activating stimuli. Together with enhanced expression of co-stimulatory molecules by B cells and/or re-localization of DCs into T cell follicular zones, this could recruit additional S1-specific T cells (including ones expressing lower levels of the 6.5 TCR) and lead to a mutually reinforcing cycle of APC and 6.5+CD4+ T cell activation and expansion, as was shown to have occurred in arthritic TS1xHACII mice. The effector mechanisms causing inflammatory arthritis could depend on co-expression of self-reactive endogenous TCE α-chains. Indeed more TS1 T cells co-expressing the 6.5 TCR and endogenous TCR □-chains underwent division following adoptive transfer into HACII than HA104 mice, reflecting the increased potency of the S1 peptide. However, even if the effector function(s) of the autoreactive CD4+ T cells that cause inflammatory arthritis in TS1xHACII mice depends on co-expression of dual TCR □-chains, the activation of these cells is critically dependent on the expression of the target peptide for the 6.5 TCR (i.e. the S1 peptide) directly by APCs, since these effector functions do not become activated in TS1xHA104 mice.

At the level of immune repertoire formation, increased deletion of autoreactive thymocytes may also contribute to the development of autoimmune disease in TS1xHACII mice by eliminating regulatory T cells. The percentages of 6.5+CD4+ splenocytes expressing a CD25+CD45RB^{int} phenotype were similar in TS1xHACII and TS1 mice. It was previously shown that thymocytes expressing the 6.5 TCR undergo little or no deletion in response to the S1 peptide in another lineage (TS1xHA28 mice), but instead undergo selection to become CD25+ CD45RB^{int} regulatory T cells (Jordan, 2000, cited above). The under-representation of these cells in TS1xHACII mice relative to both TS1xHA28 and TS1xHA104 mice suggests either that the S1 peptide fails to mediate selection of 6.5+ CD4+ CD25+ CD45RB^{int} regulatory T cells in TS1xHACII mice, or that such cells are eliminated because of enhanced thymic deletion. From a therapeutic standpoint, whether or not adoptive transfer of 6.5+CD4+ CD25+T cells can prevent arthritis in TS1xHACII mice is a possibility. However, the extended period over which arthritis develops, may complicate the development of effective protocols. In additional whether or not arthritis can be prevented in TS1xHACII mice by transfer of CD25+CD45RB^{int} T cells, the relative absence of these cells in TS1xHACII mice supports models positing that elimination of regulatory T cells can be a contributing factor in the development of autoimmune disease.

Expression of the HA by APCs is crucial to the autoimmune disease that develops in TS1xHACII mice, although it is not clear why this leads to inflammatory arthritis, rather than a more severe systemic inflammatory response such as can occur during graft-versus-host responses which similarly target APCs. Perhaps at early stages of the disease APCs in the joints undergo enhanced activation secondary to systemic immune activation. Rheumatoid synovium has been shown to be enriched in activated DCs expressing high levels of MHC class II and co-stimulatory molecules, and this might facilitate presentation of S1 as a target for 6.5+ T cells. However, anatomic features of the joints such as stress and/or poor vascularization could also be crucial to the development of a severe regional inflammatory response. Systemic over-expression of the pro-inflammatory cytokine TNF-□ induces arthritis suggesting that the joint can be highly sensitive to inflammatory responses (Keffer et al. 1991 EMBO J, 10:4025-31) even though the basis for this targeting has yet to be determined. It was also illustrated that the 6.5+ CD4+ T cells that accumulate systemically in TS1xHACII mice are hyporesponsive to antigenic stimulation (as has also been described in patients with active RA, and this can limit their capacity to mediate systemic inflammatory responses and contribute to the joint-targeting of the disease. The lung disease that develops in TS1xHACII mice can nevertheless be a consequence of the systemic activation and expansion of CD4+ T cells. TS1xHA104 mice develop a similar, albeit less severe pulmonary perivascular mononuclear cell infiltrate, and the lungs of both TS1xHACII and TS1xHA104 mice contain increased numbers of 6.5+ CD4+ T cells exhibiting activated phenotypes, consistent with previous studies showing activated CD4+ T cells trafficking to and accumulating in the lungs.

TS1xHACII mice develop an inflammatory arthritis that resembles human RA because they contain a high frequency of CD4+ T cell precursors that recognize a nominal antigen, acting as a self-antigen, when it is expressed by APCs. A similar situation may generate RA in humans in situations in which individuals carrying RA susceptibility alleles also contain a high frequency of T cell precursors that can recognize self-peptides derived from their own APCs, in a manner that is analogous to allorecognition. MHC class II molecules often contain peptides that are synthesized endogenously by APCs (Rudensky et al. 1991 Nature 353:622-7; Chicz et al. 1992 Nature 358:764-8), and such peptides could possess the same properties of direct and inducible expression in APCs as the S1 peptide exhibits in TS1xHACII mice. Alloreactive T cells also comprise a significant fraction of the pre-immune T cell repertoire, allowing RA patients expressing a polyclonal (as opposed to transgenic) T cell repertoire to contain a high frequency of autoreactive CD4+ T cells directed to self-peptide(s) expressed by APCs. Deletion of those thymocytes with high affinities for the host's own MHC:self-peptide complexes was suggested as a means to prevent autoimmunity (Robey et al. 1994 Annu. Rev. Immunol. 12:675-705).

However, the present invention demonstrates that even in the context of profound deletion of autoreactive thymocytes, expression directly by APCs makes a self-peptide arthritogenic when the CD4+ T cell repertoire is biased toward reactivity with that MHC:self-peptide complex. This suggests that the central feature in the etiology of RA is not its induction by an exogenous arthritogenic antigen, but rather the development of a CD4+ T cell response to an endogenous autoantigen, expressed by APCs, that manifests as inflammatory arthritis because of the unique environment of the joint. Since a TCR with specificity for a ubiquitously expressed (rather than tissue-specific) peptide can provoke an autoimmune disease with organ-specific manifestations, the design of effective therapies will require a fuller understanding of the parameters governing the pathologic outcomes of systemic autoreactivity, and can be facilitated by studies in TS1xHACII mice.

### EXAMPLE 1

### Transgenic Mammalian Models

A. Models Expressing an Influenza HA Polypeptide

Transgenic HA104 mice contain DNA encoding the full-length, membrane-bound polypeptide of the influenza virus A/PR/8/34 HA linked to the SV40 early region promoter/enhancer sequences (F. F. Shih et al, 1997 Int. Immunol., 9:249; M. P. Riley et al, 2000 J Immunol., 165:4870-4876). This transgenic mouse lineage expresses transgene mRNA in the thymus and in peripheral lymphoid tissues at high levels. This transgenic lineage generates reduced T cell proliferative responses relative to BALB/c mice, to the major I-E^{d}-restricted T cell determinant from the HA, termed S1. Transgene mRNA expression in tissues from HA104 mice was determined by RT-PCR analysis (Shih et al, 1997, cited above). These mice express HA diffusely, but do not synthesize the HA by their APCs.

Transgenic HA28 mice contain DNA encoding a truncated polypeptide of the influenza virus A/PR/8/34 HA linked to the SV40 early region promoter/enhancer sequences (F. F. Shih et al, 1997 cited above).

Transgenic HACII mice were generated by cloning a cDNA copy of the HA gene of influenza virus A/PR/8/34 (PR8) into the unique EcoRI site of the plasmid pDOI-5, described in detail in Kouskoffet al. 1993 J. Immunol. Methods 166:287-91, incorporated by reference herein. The use of pDOI-5 enabled the HA gene to be linked to and its expression directed by a portion of the MHC class II I-E□ gene promoter and splicing/polyadenylation signals. This linkage of the HA gene to the MHC class II promoter directs expression of the HA to B cells and CD11c+ dendritic cells. Plasmid sequences were removed and DNA was injected into fertilized BALB/c x C57B1/6 zygotes. The progeny from one founder were crossed successively with BALB/c mice. Most of the examples were performed with mice that had been backcrossed at least 5 generations with BALB/c mice. Mice of this lineage express HA endogenously by their APCs.

B. Models Expressing a T Cell Receptor

Transgenic TS1 mice express a transgene-encoded TCR specific for the major I-E^{d}-restricted determinant of PR8 HA (site 1, S1) and are described in detail in J. Kirberg et al, 1994 J. Exp. Med., 180:25-34; Riley et al, 2000, cited above; and Gerhard et al. 1991 J. Virol. 65:364-72. S1 is an 11 amino acid peptide derived from the hemagglutinin antigen of Influenza strain A/PR/8/34, and has the amino acid sequence SFERFEIFPKE (SEQ ID NO: 1).

Transgenic TS1 (SW) mice express a transgene-encoded TCR specific for a variant influenza virus, designated SW, that contains two amino acid substitutions relative to the major I-E^{d}-restricted S1 determinant of PR8 HA. The mutant S1(SW) peptide is an 11 amino acid peptide that differs from the S1 peptide in two amino acids and has the amino acid sequence SFEKFEIFPKT (SEQ ID NO: 2). The TS1(SW) TCR has an approximately 100-fold lower affinity for the S1 peptide than does the TS1 TCR (Jordan et al, 2001, cited above).

All mice were maintained in sterile microisolators.

### EXAMPLE 2

### Assay Methods

The following Examples 3-6 employ and discuss the results of the application of a number of the following procedures and protocols, which are assembled and discussed herein for ease of reference.

A. Flow Cytometry Procedures

Flow cytometry was performed on single cell suspensions isolated from the bone marrow, thymus or spleen of 10- to 16-week-old mice. For isolation of dendritic cells (DCs), spleens were injected with 2.5 ml of collagenase (6 mg/ml; Gibco-BRL) and DNAse I (0.3 mg/ml; Roche Molecular Biochemicals) in supplemented IMDM, teased apart and incubated for 1 hour at 37° C. A single cell suspension was prepared by passage through an 18.5G needle and cells were washed into supplemented IMDM with 10% fetal bovine serum (FBS).

Cells were first incubated with unlabeled anti-CD16/CD32 (2.4G2) to block Fc□ receptors and then stained with labeled antibodies. Antibodies used were: anti-1-A^{d}-FITC (AMS-32.1), anti-CD45R/B220-PE (RA3-6B2), anti-CD11c-FITC (HL3), anti-CD4-PE (GK1.5), anti-CD8-FITC (53-6.7), anti-CD25-PE (PC61), anti-CD25-FITC (PC61), anti-CD45RB-PE (16A), anti-CD69-PE (H1.2F3), anti-CD80-PE (16-10A1), anti-CD86-PE (GL1) and anti-CD44-FITC (IM7), all obtained from PharMingen (San Diego, Calif.). Anti-HA-biotin (purified from a mouse anti-HA IgG producing hybridoma) and 6.5-biotin were also used. Biotin-labeled antibodies were detected using Streptavidin-670 (Gibco BRL). Samples were analyzed on FACScan™ or FACScalibur™ flow cytometers and analyzed using CELLQuest™ software (Becton Dickenson, San Diego, Calif.). Between 50,000 and 200,000 events were collected for each sample.

B. In Vitro APC Stimulation

For in vitro B cell stimulation, splenocytes were cultured for 48 hrs in 24-well flat-bottom plates at 1×10⁶ cells/ml in supplemented IMDM+10% FBS with or without the addition of 1 μg/ml Fab2′ anti-μ (Jackson ImmunoResearch Laboratories, West Grove, Pa.) or a CD154-CD8 fusion protein, anti-CD8 antibody and IL-4 (Mandik-Nayak et al. 2000 J. Immunol. 164:1161-8 (Mandik-Nayak I)).

For in vitro DC stimulation, cells isolated from spleens as noted above were incubated for 2 hours at 37° C. on 150 mm petri dishes, after which non-adherent cells were removed and the plates were incubated overnight at 37° C. with fresh supplemented IMDM+10% FBS. Non-adherent cells were harvested the following day and examined by flow cytometry.

C. T Cell Stimulations

For measurement of S1 presentation in vitro, LN cells (5×10⁴) from TS1 mice were plated with splenocytes (5×10⁵) from HA104 mice, HACII mice, or BALB/c mice with or without S1 peptide. To measure T cell proliferative capacity, splenocytes (5×10⁴) from TS1, TS1xHA104 or TS1xHACII mice were cultured with BALB/c splenocytes (5×10⁵) and graded doses of S1 peptide or anti-CD3 mAb (Pharmingen). In all cases cultures were incubated for at 37° C. for 3 days in 200 μl of supplemented IMDM+10% FBS in flat bottomed 96-well plates, and then pulsed with 0.5 mCi/well of ³H-thymidine for 16 hrs before harvest.

For measurement of S1-specific T cell proliferation in vivo, LN cells from TS1 mice were labeled with the dye, 5-(and 6)-carboxyfluorescein diacetyl succinimidyl ester (CFSE; Molecular Probes, Eugene, Oreg.) as described (Jordan I) and transferred (˜2×10⁷/mouse i.v.) into non-tg, HACII or HA104 mice. Four days later splenocytes were isolated and examined by flow cytometry.

D. Tissue Histology

Tissues were fixed in 10% buffered formalin (Fischer Scientific, Fair Lawn, N.J.) and immobilized in paraffin. Five μm sections were cut and stained using Harris Modified Hematoxylin (Fischer Scientific) and Eosin Y (Fischer Scientific). For immunohistochemistry, spleens were submerged in OCT (VWR, West Chester, Pa.), frozen with 2-methyl butane cooled with liquid N₂, sectioned, and fixed with acetone. Sections were stained (Mandik-Nayak et al. 1997 J. Exp. Med. 186:1257-67 (Mandik-Nayak II)) with anti-CD11c-FITC (N418) and anti-CD22-biotin (Cy34.1) (Pharmingen). FITC-conjugated antibodies were detected using Streptavidin-horseradish peroxidase (HRP) (Southern Biotechnology Associates). AP and HRP were developed using the substrate Fast-Blue BB base and 3-amino-9-ethyl-carbazole (Sigma, St. Louis, Mo.) respectively.

E. Enzyme Linked Immunosorbant Assays

ELISAs were carried as described in (Reed et al., 2000 J. Exp. Med., 192:1763-1774) except that 0.05% Tween 20 (Sigma) was used in wash buffers for IgG and GPI assays and 0.05% Tween 80 (Sigma) was used in rheumatoid factor assays.

For detection of serum IgG, plates were coated with goat-anti mouse IgG (Southern Biotechnology Associates, Birmingham, Ala.) and bound antibody detected with alkaline phosphatase (AP)-conjugated goat anti-mouse IgG (Southern Biotechnology).

For detection of anti-HA antibodies, plates were instead coated with purified PR8 virus.

For rheumatoid factor assays, plates were coated with purified monoclonal mouse IgG and bound antibody detected with AP-conjugated goat anti-mouse IgM (Southern Biotechnology); positive control sera were from mice undergoing a graft-versus-host response (Dr. Terri Laufer, University of Pennsylvania).

For GPI assays, plates were coated with rabbit skeletal GPI (Schaller et al., 2001) (Sigma) and bound antibody detected with AP-conjugated goat anti-mouse IgM+IgG; positive control sera from KxB/N mice (Dr. Michael Maldonado, University of Pennsylvania). Assays for serum anti-type II collagen antibody were performed using the Arthrogen-CIA ELISA kit (Chondrex, Redmond, Wash.) according to the manufacturer's instructions.

### EXAMPLE 3

### The Development of Inflammatory Arthritis Resembling Rheumatoid Arthritis in TS1xHACII Mice

To examine how self-antigens expressed by MHC class II+ cells mediate negative selection of autoreactive CD4+ T cells, a new mouse model was generated for inflammatory arthritis (TS1xHACII mice). The transgenic mouse lineage (HACII) expressing the HA antigen under the control of an MHC class II promoter was mated with the mouse lineage (TS1), expressing a transgenic TCR specific for the major I-E^{d}-restricted determinant of HA (S1). Both lineages are described in Example 1A. Thus, TS1xHACII mice co-express the influenza virus PR8 hemagglutinin (HA) under control of a MHC class II promoter and a HA-specific MHC class II-restricted TCR. Unexpectedly, an unusual phenotype emerged. A substantial fraction of the TS1xHACII mice develop swollen joints and an inflammatory process in the lungs that are characteristic of human rheumatoid arthritis. Specifically, the symptoms shared with RA include joint swelling with bone resorption and remodeling and a perivascular mononuclear cell infiltration in the lungs accompanied by interstitial pneumonitis.

The phenotype of the TS1xHACII progeny is characterized by the development of autoimmune manifestations resembling RA. At 20 weeks, these progeny expressing both TS1 and HACII transgenes were typically smaller than littermates expressing only the TS1 or HACII transgenes. These intermated progeny frequently exhibited a hunched appearance. Ankle widths of TS1, HACII and TS1xHACII mice were measured weekly with a micrometer caliper by investigators blinded to mouse genotype and the larger of front ankles was noted. The variation between ankles was typically less than 0.2 mm. Statistical significance of differences between TS1xHACII mice and TS1 and HACII mice were determined using the Mann-Whitney test, *P≦0.05, **P≦0.01.

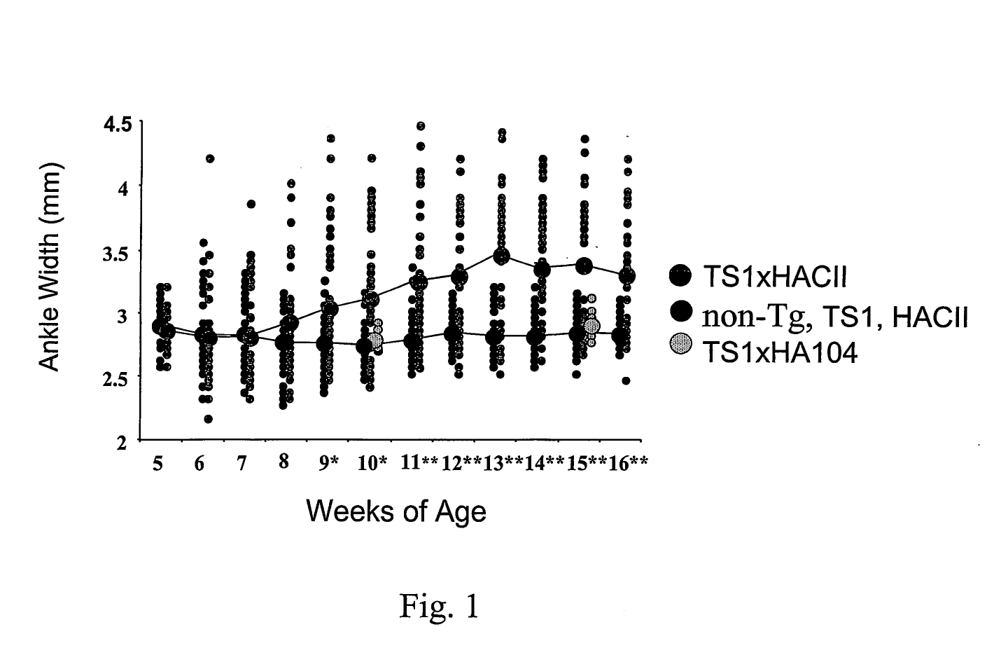

Older TS1xHACII mice often had impaired mobility and were unable to maintain their position when placed on an inclined grating, suggesting weakness in their paws. Indeed, by 10 weeks of age the wrist and ankle joints of TS1xHACII mice were significantly swollen compared with littermates carrying only the TS1 or HACII transgenes, with 69% (25/37) of 10 wk-old TS1xHACII mice exhibiting an ankle width of at least 3.4 mm. See FIG. 1. Paw swelling was almost always symmetrical, and did not resolve with age.

Further, in the TS1xHACII mice, greater than 90% of female nice exhibited overtly swollen joints by 10 weeks of age, while a lower percentage (˜60%) of males developed equivalent joint swelling.

Hematoxylin and eosin were used to stain sections of the knees from 23 week-old TS1 and TS1xHACII littermates. Bone sections, cartilage and synovium were detected at 40× magnification. A 400× magnification of the section from the TS1xHACII mouse showed bone erosion (data not shown). The histological examination of swollen joints revealed a mononuclear lymphocytic infiltrate in the synovium, substantial cartilage loss, and bone resorption. In addition, mice exhibiting severe arthritis in wrist and ankle joints had unaffected spinal joints. Thus, peripheral joints of TS1xHACII mice frequently develop a severe inflammatory arthritis similar to that occurring in RA.

Additional histopathologic examination of a variety of organs from arthritic TS1xHACII mice was performed. Hematoxylin and eosin were used to stain sections of lungs from at least 4 mice of each genotypeTS1, TS1xHACII, and TS1xHA104 at 200× magnification. Inducible HA expression was shown in MHC class II+ cells on bone marrow (BM) cells and splenocytes of non-transgenic mice, HACII mice and HA104 mice (data not shown). HACII mice have high level expression of HA on bone marrow cells and low level HA expression on splenic B cells and DCs that could be up-regulated by signals that can upregulate MHC class II expression. The lungs of the TS1xHACII mice contained an intense perivascular mononuclear cell infiltration that was in some cases accompanied by interstitial pneumonitis. Some mice also displayed evidence of lymphocytic valvulitis and myocarditis in the heart (data not shown). By contrast, there was no evidence of autoimmune or inflammatory pathology in kidney, liver, thyroid, salivary glands, pancreatic islets, or intestine, even in mice that manifested advanced inflammatory arthritis and pneumonitis (data not shown). Thus, the arthritis that develops in TS1xHACII mice is accompanied by inflammatory processes affecting the lung, as can occur in human RA, while other organs and tissues appear unaffected

### EXAMPLE 4

### Extraarticular Pathology without Arthritis in TS1xHA104 Mice

To determine how co-expression of the TS1 TCR and HA leads to joint-targeted and/or pulmonary inflammation in TS1xHACII mice, histopathologic examination was performed of tissues from TS1 mice, and from the TS1xHA104 lineage (Shih et al. 1997 cited above; and Riley et al. 2000 cited above). In the latter lineage, HA is expressed under control of the SV40 early region promoter/enhancer

The swollen joints and impaired mobility observed in TS1xHACII mice have never been observed in either TS1 or TS1xHA104 mice. No evidence of inflammation was found in joint sections made from TS1xHA104 mice. Interestingly, the lungs of TS1xHA104 mice exhibited a perivascular mononuclear cell infiltrate as was seen in TS1xHACII mice in Example 2, although it was not as severe.

Thus, pulmonary inflammation develops in both TS1xHACII and TS1xHA104 mice, but inflammatory arthritis is unique to TS1xHACII mice. Differences in the expression of the S1 peptide between the HACII and HA104 lineages must therefore be crucial to the development of inflammatory arthritis.

### EXAMPLE 5

### Direct Expression of S1 Peptide by APCs in HACII Mice

A. To examine how HACII and HA104 mice differ in their presentation of HA to CD4+ T cells, cell surface expression of HA on MHC class II+ cells was analyzed. HA transgene mRNA expression was evaluated in kidney, spleen, thymus, heart, lung, intestine and bone marrow from HA104 mice. Southern blot analyses of products obtained from RT-PCT reactions were performed using HA transgene mRNA-specific primers. Ethidium bromide stains of RT-PCR reaction products were obtained using MHC class I-specific primers. Products obtained when tail DNA was used as substrate yields a larger product with MHC class I primers, which span an intron.

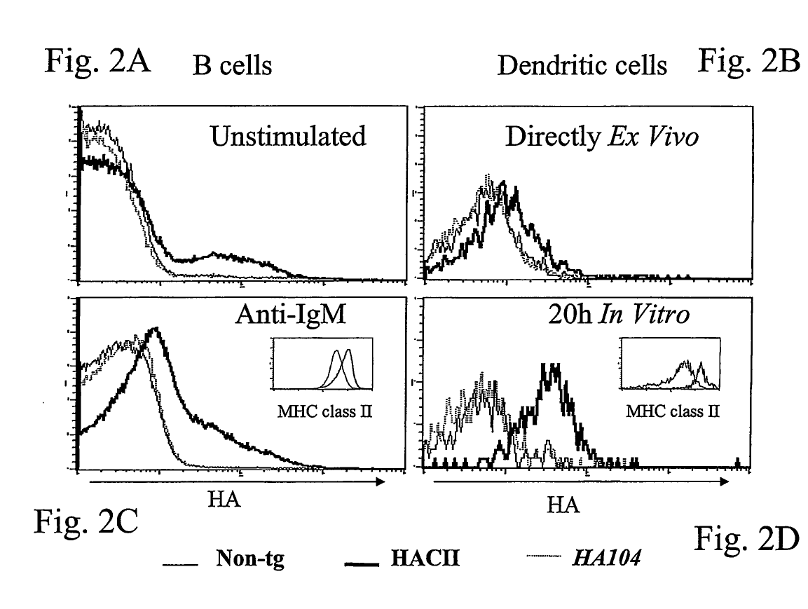

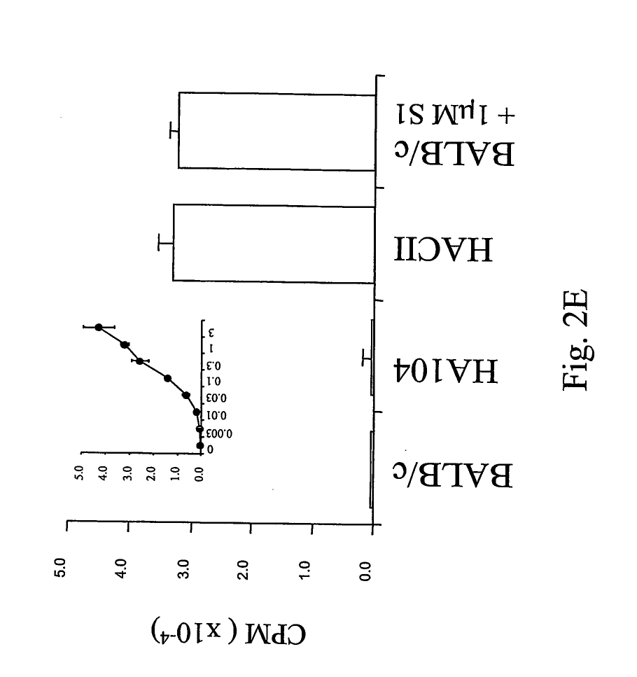

HA expression was found to increase in parallel with MHC class II on B220+ bone marrow (BM) cells. In contrast, HA was not detected on B220+ BM cells obtained either from non-transgenic (non-tg) littermates or from HA104 mice. When B220+ splenocytes from HACII mice were analyzed directly ex vivo, a small fraction were found to express cell surface HA. These HA+ cells appeared to be predominantly immature B cells based on their low levels of MHC class II and IgD. Notably, the majority of B220+splenocytes from HACII mice (but not from non-tg or HA104 mice) expressed cell surface HA following in vitro stimulation with anti-IgM, or with IL-4 and a CD154 fusion protein (FIGS. 2A-2D).

Similarly, splenic CD11c+ dendritic cells (DCs) from HACII mice expressed low levels of cell surface HA when analyzed directly ex vivo, but following overnight culture in vitro all of the CD11c+ DCs from HACII mice (but not those from non-tg or HA104 mice) expressed cell surface HA (FIGS. 2A-2D).

Although HA was undetectable by flow cytometry in HA104 mice, HA transgene mRNA was detected in all tissues examined. Together, these data indicate that HA expression is targeted to MHC class II+ cells in HACII mice, and that stimuli that increase MHC class II expression in B cells and DCs also lead to increased HA expression.

B. To compare HACII and HA104 mice for their abilities to process and present the S1 peptide to CD4+ T cells, the ability of splenocytes from HACII, HA104 or non-tg mice to induce proliferation of S1-specific T cells from TS1 mice in vitro was assessed. See, FIG. 2E. Whereas HA104 and non-tg splenocytes failed to induce proliferation of TS1 T cells, splenocytes from HACII mice induced a similar degree of proliferation to that induced by BALB/c splenocytes in the presence of 1 μM S1 peptide.

C. Presentation of the S1 peptide in vivo was then examined by labeling LN cells from TS1 mice with CFSE according to the method of Lyons and Parish 1994 J. Immunol. Methods 171:131-137, followed by intravenously injecting the cells into HACII, HA104 or non-tg mice. Three independent experiments were performed. S1-specific CD4+ T cells were detected with the anti-clonotypic antibody 6.5 (Kirberg, 1994, cited above) and underwent division in both HACII and HA104 mice. More rounds of division occurred in HACII mice.

The ability to detect proliferation of TS1 T cells after transfer into HA104 mice contrasts with the in vitro proliferation studies, and likely reflects the greater sensitivity of detection afforded by analyzing proliferation of TS 1 T cells in vivo. Greater numbers of CD4+ T cells expressing low levels of the 6.5 clonotype underwent division in HACII than in HA104 mice. These low levels of the 6.5 clonotype are the result of allelic inclusion of endogenous TCR □-chains (Padovan et al. 1993 Science 262:4224 and Heath et al. 1995 Eur. J. Immunol. 25:1617-23), since all CFSE-labeled CD4+ T cells expressed similar levels of the transgenic Vβ8.2 β chain.

These data indicate that the S1 peptide is processed from the HA and presented by I-E^{d} molecules in both HACII and HA104 mice, but that expression directly by APCs makes S1 a more potent stimulator of S1-specific CD4+ T cells in HACII than HA104 mice.

### EXAMPLE 6

### Endogenous Activation of Autoreactive CD4+ T Cells in TS1xHACII Mice

Flow cytometric analyses of thymocytes and splenocytes from TS1xHACII and TS1xHA104 mice were performed to examine how differences in the expression of the S1 self-peptide affect the development of S1-specific CD4+ T cells.

A. Thymic CD4/CD8 profiles were obtained using flow cytometry from TS1, TS1xHACII and TS1HA104 mice. Thymi were stained with anti-CD4 and anti-CD8. Mean numbers of CD4+CD8+DP, CD4SP and CD8SP cells +/−standard deviation are shown in the following Table I.

TABLE I

| | Mean Numbers of Stained Cells ± S.D. | | |
|---|---|---|---|
| MICE | CD4 + CD8 + DP | CD4SP | CD8SP |
| TS1 | 15.4 ± 8.9 | 36.3 ± 22.0 | 1.0 ± 0.7 |
| TS1XHACII | 7.9 ± 4.6 | 9.1 ± 7.2 | 1.5 ± 0.8 |
| TS1xHA104 | 5.8 ± 2.5 | 23.0 ± 8.4 | 0.9 ± 0.4 |

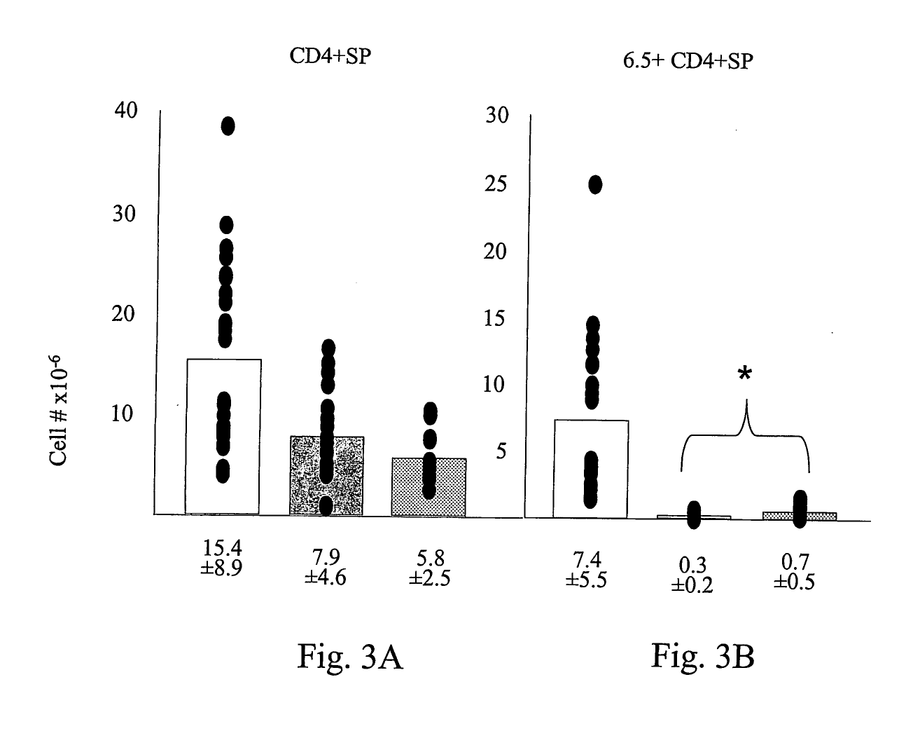

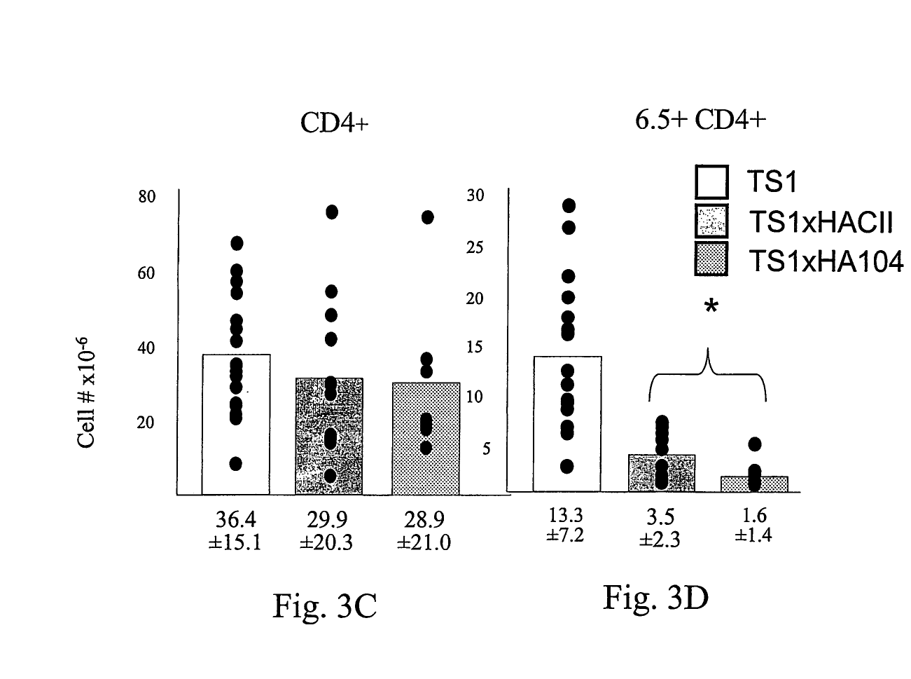

As is seen in the results of FIGS. 3A-3D, the frequencies of CD4+ CD8+ double positive (DP) and CD4+ single positive (SP) thymocytes were reduced in both TS1xHACII and TS1xHA104 mice relative to TS1 mice. Moreover, the frequencies of 6.5+ CD4+ SP thymocytes in TS1xHA104 and TS1xHACII mice were reduced on average to 9% and 4%, respectively, of TS1 mice. Interestingly, whereas 6.5+ CD4+ SP thymocytes were significantly less abundant in TS1xHACII than TS1xHA104 mice; the reverse was true for splenocytes. TS1xHACII mice contained twice as many 6.5+CD4+ splenocytes as TS1xHA104 mice, and a third as many as TS1 mice (in which there is no S1-mediated thymic deletion).

Thus, targeting expression of the S1 peptide to MHC class II+ cells increased the deletion of S1-specific thymocytes in TS1xHACII mice even above the substantial deletion that occurs in TS1xHA104 mice, consistent with the preceding Examples showing that S1 is a more potent self-peptide in HACII than HA104 mice. However, direct expression by APCs also leads to increased frequencies of 6.5+CD4+ splenocytes in TS1xHACII mice.

B. Since 6.5+CD4+ T cells appeared to have expanded in the periphery of TS1xHACII mice relative to TS1xHA104 mice, these cells were examined for evidence of endogenous activation. The endogenous activation of S1-specific CD4+ T cells in TS1xHACII mice was measured by generating histograms of the expression of CD25, CD45RB, CD69 and CD44 by 6.5+CD4+ splenocytes from TS1 mice, TS1xHACII and TS1xHA104 mice. The histograms were gated on CD4+ 6.5− or 6.5+ cells. Histograms of 6.5+ T cells from TS1xHACII and TS1xHA104 mice were scaled to approximate the number of events shown from TS1 mice. The mean percentages were obtained from at least 7 mice per genotype of cells in the region and are reported in the following Table II.

TABLE II

| | | Cells in Mean Percentages | | |
|---|---|---|---|---|
| Marker | Gate | TS1 | TS1xHACII | TS1xHA104 |
| CD25 | 6.5+ gated | 11.1 ± 1.3 | 10.7 ± 4.3 | 46.7 ± 7.9 |
| | 6.5− gated | 10.4 ± 0.7 | 20.0 ± 5.5 | 13.5 ± 1.5 |
| CD69 | 6.5+ gated | 10.0 ± 1.1 | 49.7 ± 10.3 | 28.3 ± 3.1 |
| | 6.5− gated | 10.6 ± 2.8 | 17.0 ± 3.2 | 11.1 ± 0.6 |
| CD45RB | 6.5+ gated | 10.2 ± 1.0 | 76.4 ± 12.2 | 35.6 ± 2.5 |
| | 6.5− gated | 13.9 ± 1.9 | 27.2 ± 3.9 | 20.5 ± 1.7 |
| CD44 | 6.5+ gated | 25.6 ± 3.8 | 57.2 ± 10.9 | 41.6 ± 9.9 |
| | 6.5− gated | 27.2 ± 5.9 | 39.6 ± 17.3 | 29.4 ± 9.5 |

The frequencies of 6.5+ CD4+ cells expressing cell surface phenotypes indicating prior antigenic experience (high levels of CD69 and CD44 and low levels of CD45RB) were higher in TS1xHACII than TS1xHA104 mice.

B. The endogenous activation of S1-specific CD4+ T cells in TS1xHACII mice was measured by generating histograms of the expression of CD45RB and CD25 on 6.5+CD4+ splenocytes from TS1xHA104, TS1xHA28, TS1xHACII and TS1 mice. The percentage of cells which express a CD25^{hi} CD45RB^{int} phenotype were 35.5% for TS1xHA104, 24.6% for TS1xHA28, 9.1% for TS1xHACII and 5.9% for TS1 mice. These data are representative of at least 3 mice of each genotype. The CD4+ cells expressing these activated phenotypes were enriched in the 6.5+ versus 6.5− subsets. However, few of the 6.5+ CD4+ splenocytes from TS1xHACII mice were CD25+, whereas nearly half of the 6.5+ CD4+ splenocytes from TS1xHA104 mice were CD25+ CD45RB^{int}. This phenotype is notable because it resembles that of 6.5+ CD4+ CD25+ regulatory T cells that develop in response to S1 peptide in another lineage, TS1xHA28 mice (Jordan et al., 2000 Eur. J. Immunol, 30:136-44). Indeed, while many of the 6.5+ CD4+ splenocytes from TS1xHACII mice were CD45RB^{low}, the percentage of CD25+ CD45RB^{int} was similar to TS1 mice which lack the S1 peptide that induces thymic selection of 6.5+ CD4+ CD25+ regulatory T cells (Jordan et al., 2001 Nat. Immunol. 2:301-6). Thus, 6.5+ CD4+ splenocytes from TS1xHACII mice exhibit evidence of endogenous activation, although those expressing a phenotype characteristic of regulatory T cells (CD25+CD45RB^{int}) are severely under-represented.

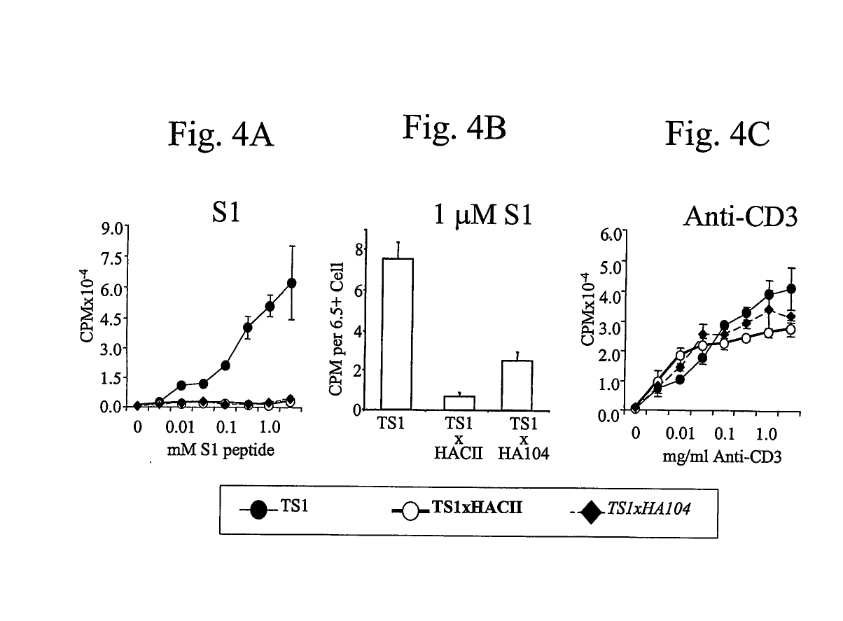

C. The proliferative capacity of the 6.5+CD4+ T cells that had accumulated in the spleens of TS1xHACII mice was studied by evaluating their ability to proliferate in vitro when incubated with S1 peptide and irradiated splenocytes from BALB/c mice. Splenocytes from TS1xHACII mice exhibited much lower proliferative responses to S1 peptide than did TS1 splenocytes, even when adjusted for their differing frequencies of 6.5+CD4+ cells. See FIGS. 4A-4C.

Splenocytes from TS1xHA104 mice were also less responsive to S1 peptide than were TS1 splenocytes, although they were more responsive on a per 6.5+ cell basis than were splenocytes from TS1xHACII mice. By contrast, responses to stimulation with anti-CD3 were similar in all mice, indicating that only S1-specific T cells in TS1xHACII and TS1xHA104 mice were hyporesponsive to antigenic stimulation. Accordingly, the 6.5+ CD4+ T cells that accumulate systemically in arthritic TS1xHACII mice have undergone endogenous activation, but exhibit an anergic/exhausted phenotype that has been described in other systems where autoreactive CD4+ T cells experience chronic exposure to a self-antigen. These observations are notable in light of studies showing that CD4+ T cells obtained from patients with active RA exhibit activated phenotypes, yet appear anergic when analyzed for their proliferative responses in vitro (see, e.g., Ali, M., et al. 2001 J. Clin. Invest., 107:519-28).

### EXAMPLE 7

### Systemic APC Activation in TS1xHACII Mice

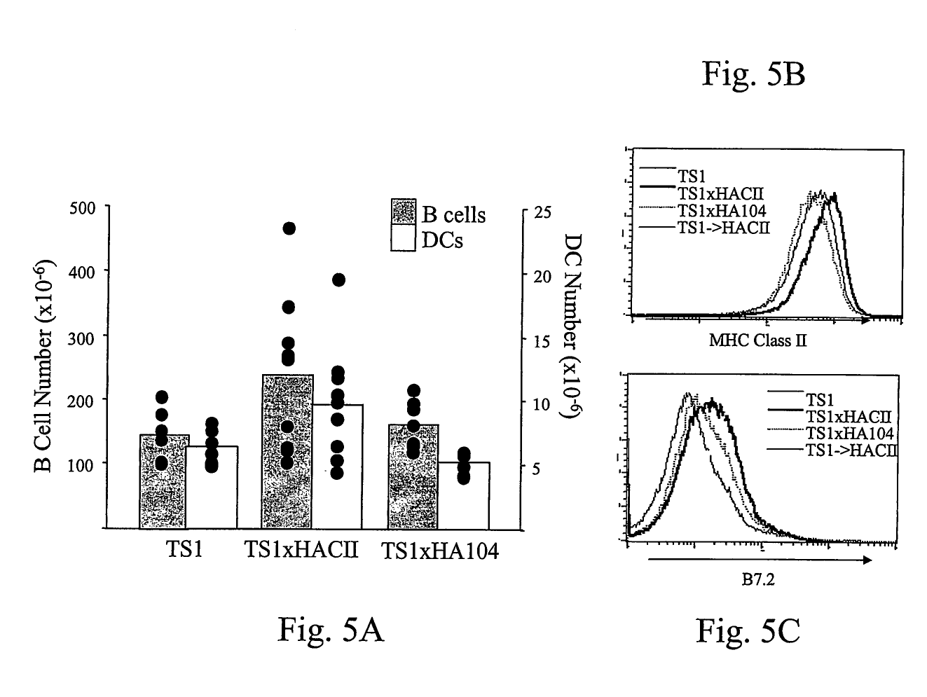

Splenic MHC class II+ cells from TS1xHACII, TS1xHA104 and TS1 mice were examined for evidence of expansion and/or activation. As illustrated in FIG. 5A, there were significantly increased numbers of B cells in the spleens of TS1xHACII mice relative to both TS1 and TS1xHA104 mice.

As illustrated in FIGS. 5B and 5C, the splenic B220+ cells from TS1xHACII mice appeared to be activated based on elevated levels of MHC class II and CD86 (but not CD80), although their levels were not as high as were found on B220+cells from HACII mice four days after adoptive transfer of S1-specific CD4+ T cells from TS1 mice. MHC class II and CD86 levels were also slightly but consistently higher on B220+ cells from TS1xHA104 than TS1 mice.

Splenic sections from TS1xHACII, TS1xHA104 and TS1 mice were stained with anti-CD22 designating B cells and anti CD11C, designating CDs. Sections representative of 3-7 mice per genotype were examined at 25× magnification (not shown). CD11c+ DCs from TS1xHACII, TS1xHA104 and TS1 mice were indistinguishable with respect to their levels of MHC class II, CD80 and CD86. However, splenic CD11c+ DCs were present in significantly larger numbers in TS1xHACII mice (FIG. 5A), and immunohistologic examination revealed that many were located in the T cell zones of splenic follicles. By contrast, CD11c+ cells in TS1 mice were mostly clustered in the bridging channels between the T cell zone and the red pulp. TS1xHA104 mice exhibited an intermediate phenotype, with many DCs located in the bridging channels and some located in the T cell zone. Follicular re-localization of DCs to the T cell zones occurs during CD4+ T cell responses to foreign antigens, although in those cases the DCs typically exhibit increased levels of MHC class II and CD86, Interestingly, re-localization of DCs expressing an “immature” cell surface phenotype to the T cell zones of splenic follicles, which was observed in TS1xHACII mice, also occurs in autoimmune lpr/lpr mice (Fields et al. 2001 J. Immunol. 167:2370-8), and could facilitate the interaction of DCs with autoreactive CD4+ T cells.

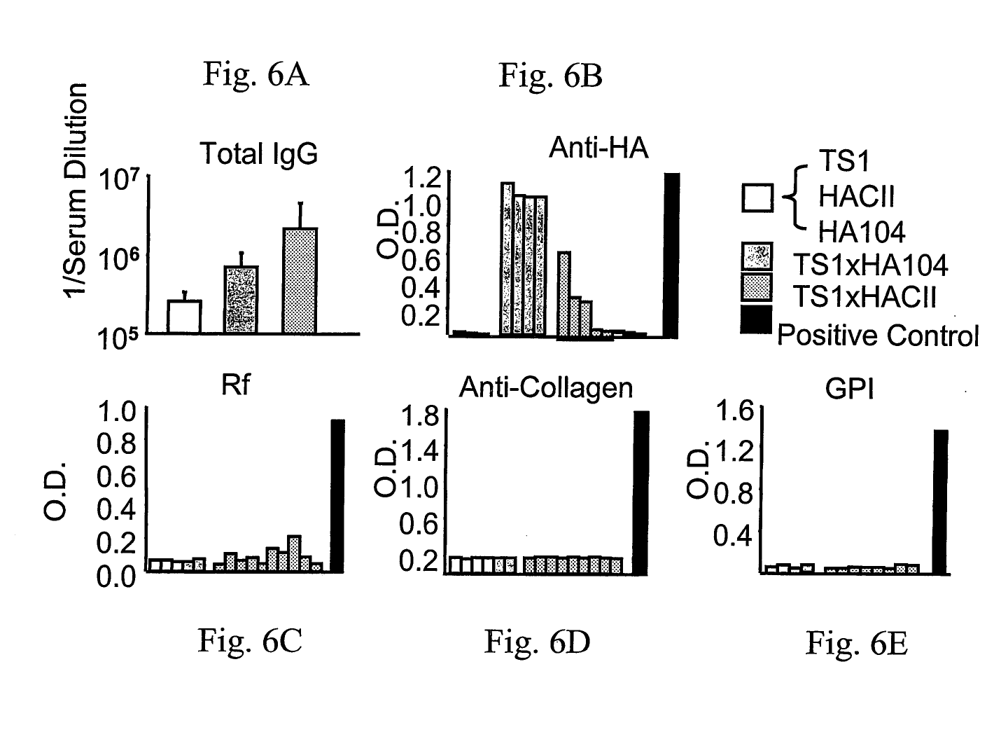

The levels of total serum IgG in the different mice paralleled the activation status of their APCs. As shown in FIGS. 6A-6E, serum IgG levels were elevated 8.1- and 2.6-fold, respectively, in TS1xHACII and TS1xHA104 mice relative to TS1 mice. Although total IgG levels were higher in sera from TS1xHACII mice, the levels of HA-specific IgG antibody were lower than in TS1xHA104 mice. As previously demonstrated, the high levels of HA-specific serum IgG in TS1xHA104 mice demonstrate that the 6.5+CD4+ T cells that are present provide cognate help for HA-specific B cells that evade deletion (Reed et al. 2000, cited above). The reduced levels in TS1xHACII mice likely reflect more efficient deletion of HA-specific B cells induced by expression of HA on developing B cells in the BM.

Sera from TS1xHACII mice was also examined for the presence of antibodies to self-antigens that have previously been associated with inflammatory arthritis (FIGS. 6A-6D). TS1xHACII mice did not contain elevated levels of serum antibody to IgG (rheumatoid factor), collagen II or GPI, indicating that these anti-self specificities are not required for the development of inflammatory arthritis.

### EXAMPLE 8

### Low Penetrance Mammalian Model

To generate a mammalian animal model displaying low penetrance of autoimmune disorder, progeny of two transgenic animals were produced. Transgenic TS1(SW) mice having a low intrinsic affinity for the S1 peptide were intermated with the HACII transgenic mice to produce low penetrance models expressing the phenotype in about 12% of the progeny. Comparisons of these models were made with progeny of TS1(SW)x HA28, TS1(SW)xHA104, and TS1(SW)x HA12.

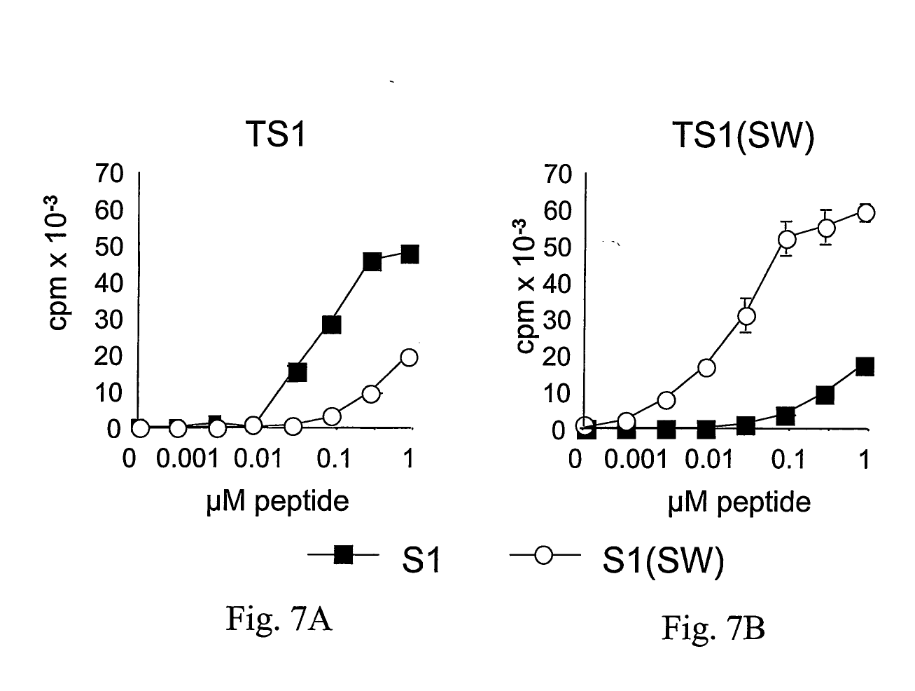

In one experiment, the binding affinities of CD4+ T cells of T cell receptor transgenic mice, TS1(SW) for the S1 peptide (SEQ ID NO: 1) and the S1(SW) peptide (SEQ ID NO: 2) were determined. As indicated in the graph of FIG. 7, the TS1(SW) T cell receptor has a high affinity for the S1(SW) peptide (i.e., about half maximal stimulation at 0.05 micromolar peptide) and a low affinity for the S1 peptide (i.e., about 100-fold lower at half maximal stimulation at greater than 1 micromolar).

The TS1(SW) T cell receptor has lower affinity for S1 shown by its needing around 30-fold higher concentrations of S1 peptide in order to reach half-maximal stimulation measured as proliferation in response to graded doses of the S1 peptide.

In histological studies similar to those described in Example 3, thymocytes expressing the TS1(SW) TCR did not develop into CD4+ CD25+ T cells in TS1(SW)xHA28 mice. They also failed to undergo selection to become CD25+T cells in TS1(SW)x HA12, TS1(SW)xHA104, and TS1(SW)xHACII mice. There was modest and then substantial deletion of TS1 (SW) thymocytes in the latter two sets of mice, but their low affinity for S1 appears to preclude their development into CD4+ CD25+ T cells in response to S1 peptide. Thus, specificity for a single peptide can direct thymocytes bearing an autoreactive TCR to undergo selection to become regulatory CD4+ CD25+ T cells.

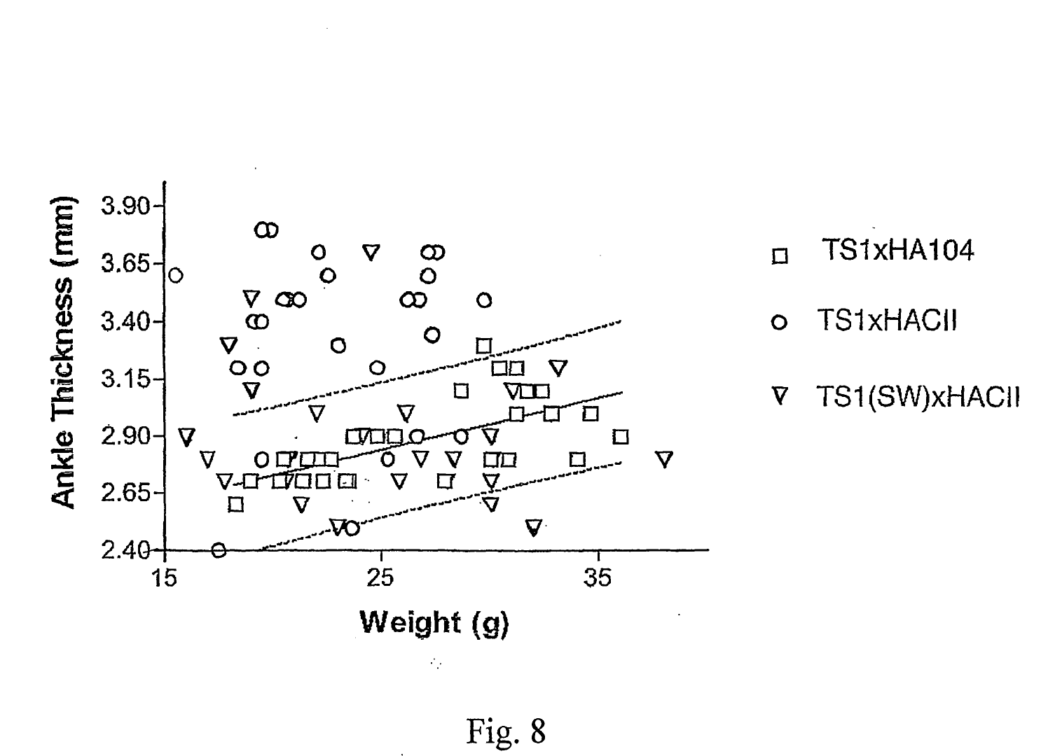

In a further phenotypic analysis, ankle thickness and weight were measured in more than 8 week-old mouse models, in which ankle thinkness results from the development of inflammatory arthritis. As shown in the results of FIG. 8, TS1xHA104 mice are used as control mice that do not develop arthritis. The majority of the TS1xHACII mice exhibit ankle widths greater than the 95% prediction interval for TS1xHA104 mice. The age-matched cohort of TS1(SW)xHACII mice show only 4 mice exhibiting significant ankle swelling indicative of the development of inflammatory arthritis as evidenced by immunohistochemical analyses of the joints. These data show that the overall avidity of the CD4+ T cell response to the S1 peptide can determine the degree of penetrance for inflammatory arthritis in these mouse models.

All publications cited in this specification are incorporated herein by reference herein.

1. A method for generating a non-human mammalian model of an autoimmune disorder, said method comprising the steps of:
(a) producing intermated progeny of a first and a second transgenic non-human mammal of the same species, wherein said first mammal expresses a major histocompatibility (MHC) class II-restricted T cell receptor (TCR) and said second mammal expresses a selected peptide that binds to said TCR, which selected peptide is selectively expressed by MHC class II positive antigen presenting cells (APC) of said second mammal; and
(b) selecting from said progeny those mammals that co-express said TCR and said selected peptide,
wherein said selected progeny develop an autoimmune disorder.

2. The method according to claim 1, wherein said model is a high penetrance model of said disorder and wherein said selected peptide is a naturally-occurring, recombinant or synthetic MHC class II-restricted T cell determinant that specifically binds with high affinity to said TCR.

3. The method according to claim 2, wherein at least 50% of said progeny before selection develop said autoimmune disorder.

4. The method according to claim 2, wherein said TCR is the TS1 TCR and said determinant is A/PR/8 hemagglutinin (HA) peptide S1.

5. The method according to claim 4, wherein said first mammal is a transgenic mouse that expresses MHC class II-restricted TCR with high affinity for an A/PR/8 hemagglutinin (HA) peptide S1 and said second mammal is a transgenic mouse that expresses DNA encoding the influenza A/PR/8 HA peptide S1 operably linked to a functional fragment of the MHC class II I-Eα promoter.

6. The method according to claim 1, wherein said model is a low penetrance model of said disorder and wherein said selected peptide is a naturally-occurring, recombinant or synthetic protein or peptide fragment that binds with low affinity to said TCR.

7. (canceled)

8. The method according to claim 6, wherein said TCR is the TS 1 (SW) TCR and said peptide is A/PR/8 hemagglutinin (HA) peptide S1.

9. The method according to claim 8, wherein said first mammal is a transgenic mouse that expresses a MHC class II-restricted TCR with high affinity for an synthetic mutant S1 analog of A/PR/8 HA, but with low affinity for the native A/PR/8 HA S1 peptide; and wherein said second mammal is a transgenic mouse that expresses DNA encoding the native influenza A/PR/8 HA S1 peptide operably linked to a functional fragment of the MHC class II I-Eα promoter.

10. The method according to claim 9, wherein said mutant analog is an HA peptide SEQ ID NO: 2 differing by two amino acids from said A/PR/8 HA S1 peptide SEQ ID NO: 1.

11. The method according to claim 1, wherein in said second mammal a first nucleic acid sequence encoding said selected peptide is operably linked to a second nucleic acid sequence that directs expression of said first nucleic acid sequence selectively to MHC class II positive cells.

12. The method according to claim 11, wherein said second nucleic acid sequence encodes the MHC class II E-α gene promoter, non-MHC class II sequences involved in expression of the invariant chain, non-MHC class II H2-M promoter, the Dec205 promoter and the Cd11c promoter.

13. (canceled)

14. The method according to claim 1, wherein said autoimmune disorder is inflammatory arthritis.

15. The method according to claim 14, wherein said disorder is characterized by inflamed joints with bone resorption, mononuclear cell infiltrates and pannus formation.

16. (canceled)

17. A non-human mammalian model of an autoimmune disorder, produced by the method of claim 1.

18-19. (canceled)

20. A transgenic non-human mammal that expresses a major histocompatibility (MHC) class II-restricted T cell receptor (TCR) and expresses a selected peptide that binds to said TCR, which selected peptide is selectively expressed by MHC class II positive antigen presenting cells (APC), wherein said mammal develops the phenotypic symptoms of an autoimmune disorder.

21-25. (canceled)

26. A recombinant mammalian cell containing at least one transgene comprising a first nucleic acid sequence that encodes a major histocompatibility (MHC) class II-restricted T cell receptor (TCR) operably linked to regulatory sequences directing its expression; and a second nucleic acid sequence that encodes a selected peptide that binds to said TCR, operably linked to a sequence that directs expression of said selected peptide selectively to MHC class II positive antigen presenting cells (APC).

27-28. (canceled)

29. A method for producing a transgenic non-human mammalian model of an autoimmune disorder, said method comprising introducing at least one transgene comprising a first nucleic acid sequence that encodes a major histocompatibility (MHC) class II-restricted T cell receptor (TCR) operably linked to regulatory sequences directing its expression; and a second nucleic acid sequence that encodes a selected peptide that binds to said TCR, operably linked to a sequence that directs expression of said selected peptide selectively to MHC class II positive antigen presenting cells (APC) into a fertilized egg, wherein said egg is transplanted into a pseudopregnant mammal and developed to term, and wherein said at least one transgenic offspring contains said transgene and is bred to form a transgenic mammal having said autoimmune disorder.

30-32. (canceled)

33. A cell culture comprising cells derived from tissues of a transgenic non-human mammal of claim 20.

34. A method of screening a compound for the ability to effect symptoms of an autoimmune disorder, comprising the steps of:
(a) administering a test compound to a mammalian model of autoimmune disorder selected from the mammal of claim 20; and
(b) comparing the severity of said symptom in the mammalian model (a) to a control mammal to which said test compound was administered.

35. (canceled)

36. A method of identifying a gene product responsible for the development of autoimmune disorders comprising the steps of:
identifying expression levels of a gene product of a mammalian model of autoimmune disorder selected from the mammal of claim 20;
comparing expression of said gene product with the expression of the same or analogous gene product in a control mammal; and
determining a difference in the expression of said gene product, wherein the absence, upregulation or downregulation of said gene product between said model and said control.

37-38. (canceled)

39. A method for identifying a biochemical marker of an autoimmune disorder comprising:
comparing the T cells or MHC class II positive APC of a mammalian model of claim 20 with a high genotypic penetrance of said disorder with the T cell of a said mammalian model with a low genotypic penetrance of said disorder; and
identifying a biochemical marker present on T cells or MHC class II positive APC of one model that is not present the T cells or MHC class II positive APC of the other model;
wherein the presence of said marker on said high penetrance model and absent on said low penetrance model or the absence of said marker on said high penetrance model and its presence on said low penetrance model is an indicator of a high likelihood of the development of said autoimmune disorder.

40-45. (canceled)
